(19) United States
(12) Patent Application Publication
(10) Pub. No.: US 2026/0174403 A1
(43) Pub. Date: Jun. 25, 2026

(54) DENTAL RADIOGRAPHY METHOD WITH ENHANCED MATERIALS CHARACTERIZATION

(71) Applicant: Carestream Dental LLC, Atlanta, GA (US)

(72) Inventors: Jay S. SCHILDKRAUT, Rochester, NY (US); Subramanyan KRISHNAMOORTHY, Penfield, NY (US); Jean-Marc INGLESE, Bussy-Saint-Georges (FR); Vincent LOUSTAUNEAU, Fontenay sous Bois (FR); Yoann PAVIA, Paris (FR); Edward R. SHELLARD, Atlanta, GA (US); Stephane VARLET, Chenehutte (FR); Stephane ALRIC, Paris (FR)

(21) Appl. No.: 18/897,037

(22) PCT Filed: Apr. 3, 2023
(86) PCT No.: PCT/US2023/017316

§ 371 Date: Sep. 26, 2024

Related U.S. Application Data
(60) Provisional application No. 63/327,676, filed on Apr. 5, 2022.

(51) Int. Cl. A61B 6/02; A61B 6/00; A61B 6/46; A61B 6/51

(52) U.S. Cl. CPC A61B 6/025; A61B 6/461; A61B 6/482; A61B 6/51

(57) ABSTRACT

A method for forming radiographic images of oral anatomy of a subject exposes the subject to radiation along a radiation path. At each position on a detector array in the radiation path, the number of photons received from the radiation through the subject are counted, wherein the detector maintains a first photon count for photons having a first energy above a first threshold energy value and at least a second photon count for photons having a second energy above a higher threshold energy value. One or more materials in the mouth of the subject are identified by performing material decomposition processing, according to two or more basis materials, and distinguishing between natural oral anatomy features and fabricated materials in the mouth of the subject according to the material decomposition processing. Conditioned image content enhances the distinction between natural oral anatomy features and fabricated material according to the material decomposition.

### TECHNICAL FIELD

The disclosure relates generally to dental radiography and more particularly to the use of spectral radiography in dental and cephalometric imaging for characterization and differentiation of materials in image content of a sample and for enhanced visualization of intraoral features according to their corresponding materials.

### BACKGROUND

Conventional dental x-ray imaging is acknowledged to be a valuable tool for showing the internal condition of teeth and underlying support structures as well as aiding in various procedures and in the installation and assessment of various dental appliances, including crowns, implants, and other devices. Dental X-rays are highly useful not only in support of particular procedures, but can also serve as useful tools in monitoring intraoral health as part of periodic imaging procedure.

The intraoral environment presents significant challenges for radiographic characterization. The mouth includes a range of materials and tissue types, ranging from fluids and soft tissue to bone and tooth materials of variable densities. In addition, various dental treatments and procedures can introduce a number of diverse materials, ranging from dental fillings to crowns, implants and other prosthetics, and including materials used in endodontics, periodontics, and other specialties. Within, surrounding, or even replacing various intraoral structures, a range of materials, including various metals, ceramics, plastics, composites, and other materials can be temporarily or permanently installed in the midst of surrounding tooth and supporting structures.

The highly compact conditions of the patient's mouth, concerns for patient comfort and safety during imaging, constraints on detector array size, placement, and support, and the need for working with or around various prosthetic devices, compounded by the need to minimize dose to the lowest possible levels, can make it particularly difficult to acquire radiographic images that provide the information desired by the practitioner, at the optimal energy levels needed for providing information related to intraoral features and the overall condition of the patient.

To further compound the problem, the imaging content itself for different types of tissue and other materials depends, in large part, on the energy level of the radiation that is provided, so that lower energy levels best suited for characterizing gum tissue, for example, yield disappointing or even unusable results for imaging tooth or bone structure or features formed of metal or implant materials.

X-ray imaging provides image content according to attenuation coefficients of the image features that lie in the path of the x-ray beam. Using standard radiography, it can be very difficult to ascertain material composition of the imaged anatomy except where there is only one predominant type of tissue or material.

The capability to distinguish types of materials would be highly beneficial in a number of dental applications. It can be useful for the dental practitioner to have the capability to separately visualize features such as fillings of metal or synthetic materials apart from, as well as within, the surrounding tooth structure. This capability would also enable virtual material removal, so that structures of different materials could be more readily segmented from each other, then reconstructed and displayed separately. For example, procedures for root canal treatment would benefit from improved segmentation of soft tissue from surrounding structures, allowing the practitioner to visualize the pulp chamber walls separately from the gutta-percha filling. Metal crown segmentation for endodontic treatment could also take advantage of enhanced imaging capabilities for materials, with virtual material removal that allows more careful analysis of the underlying bone structure, separate from the crown. Osseointegration assessment of implant preparation and condition can be improved by virtual material removal, allowing the ability to more clearly distinguish types of materials from each other.

Incomplete records and other factors can make it difficult for the practitioner to determine what materials may have been employed by others in previous treatment of a patient. Current practices can require painstaking tooth-by-tooth observation in order to assess and analyze patient history.

In these and other applications, the practitioner would clearly benefit from being able to visually separate the materials within the mouth in order to more properly assess the overall condition and disposition of various naturally occurring and fabricated features and structures. Without this capability, the dentist may need to interpret some X-rays using an amount of guesswork and approximation, risking errors in judgement and potentially jeopardizing safe and effective patient care.

Thus, it can be appreciated that there would be benefits to dental radiography tools and techniques that enhance the capability of the practitioner to separate and view the various materials and features of the patient's mouth according to material properties.

### SUMMARY

An object of the present disclosure is to advance the art of dental radiography. An embodiment of the present disclosure particularly addresses the need for distinguishing particular materials within the patient's mouth and using this knowledge for improved characterization of intraoral features and for allowing virtual material removal for improved visibility of natural and synthetic structures.

Another object of this application is to address, in whole or in part, at least the foregoing and other deficiencies in the related art.

It is a related object of this application to provide, in whole or in part, at least the advantages described herein.

These objects are given only by way of illustrative example, and such objects may be exemplary of one or more embodiments of the application. Other desirable objectives and advantages inherently achieved by the disclosed methods may occur or become apparent to those skilled in the art. The invention is defined by the appended claims.

In addition, it can be appreciated that there are benefits to a dental practitioner in having an enhanced capability to measure the dimensions of both tissue and added materials. Dimensional metrics can relate to measures of linear extent (such as height, width, and depth), circumference, surface area, or volume, for example.

According to one aspect of the disclosure, there is provided a method for forming radiographic images of oral anatomy of a subject comprising:
a) exposing the subject to radiation along an x-ray radiation path;
b) obtaining image content by counting the number of photons received, on a detector array in the radiation path, from the radiation through the subject at each of a plurality of positions,
wherein the detector maintains a first photon count for photons having a first energy above a first threshold energy value and further maintains at least a second photon count for photons having a second energy above a higher threshold energy value;
c) identifying one or more materials in the mouth of the subject by performing material decomposition processing, according to two or more basis materials that have been previously characterized according to the at least first and second photon counts, and distinguishing between natural oral anatomy features and fabricated materials in the mouth of the subject according to the material decomposition processing;
and
d) displaying conditioned image content that enhances the distinction between the identified natural oral anatomy features and fabricated materials in the mouth according to the material decomposition.

### BRIEF DESCRIPTION OF THE DRAWINGS

The foregoing and other objects, features, and advantages of the invention will be apparent from the following more particular description of the embodiments of the disclosure, as illustrated in the accompanying drawings.

The elements of the drawings are not necessarily to scale relative to each other.

FIG. 1 is a schematic diagram of a dental imaging system for performing an x-ray scan of a patient for generating a 3D volume reconstruction of a portion of the patient according to the present disclosure.

FIG. 2A is a schematic diagram showing the photon-counting sequence.

FIG. 2B is a schematic diagram showing the photon-counting sequence with multiple threshold values.

FIG. 3 is a graph that shows an arrangement of energy bins defined over the energy spectrum.

FIG. 4A is a graph showing exemplary linear attenuation characteristics for two different materials.

FIG. 4C is a graph showing exemplary linear absorption characteristics for two different materials.

FIG. 4D is a graph showing curves of relative coefficients of attenuation for water, bone, and calcium.

FIG. 5A is a logic flow diagram that shows an overview of the imaging process according to an embodiment of the present disclosure.

FIG. 5B is a logic flow diagram that shows the setup step in greater detail.

FIG. 5C shows some of the variations possible for an acquisition step using the procedures of the present disclosure.

FIG. 5D shows aspects of a materials characterization step.

FIG. 5E shows logic flow for parts of the processing and assessment steps according to an embodiment of the present disclosure.

FIG. 5F is a flow diagram that shows a sequence of steps for acquisition and processing of image content for virtual material removal according to an embodiment of the present disclosure using spectral imaging.

FIG. 6A shows, by way of example, the use of scout image content for populating a dental chart for the patient.

FIG. 6B shows other methods that can be used for editing bin definition thresholds on a user interface associated with the x-ray imaging system.

FIG. 6C shows a sequence for generating material-separated projection images.

FIG. 6D shows a schematic of a sequence for processing material-separated projection images.

FIG. 7A shows a schematic for a dental phantom that is scanned using spectral x-rays in order to generate 3D volume reconstructions.

FIG. 7B shows an attenuation reconstruction for gutta percha material in the dental phantom of FIG. 7A.

FIG. 7C shows a 3D volume reconstruction obtained using HA (hydroxyapatite) as a basis material.

FIG. 7D shows a 3D volume reconstruction obtained using titanium (Ti) as a basis material.

FIGS. 8A and 8B show examples for reconstructions from a tooth having gutta-percha material.

FIG. 8C compares axial views from the reconstruction of FIGS. 8A and 8B for conventional attenuation, soft tissue density, and combined soft tissue (ST) and HA density.

FIG. 8D compares coronal views from the reconstruction of FIGS. 8A and 8B for conventional attenuation, soft tissue density, and combined ST and HA density.

FIG. 9A shows an arrangement of a titanium post that lies partially within an SiO₂ crown for a dental phantom.

FIG. 9B shows a conventional reconstruction based on attenuation for the features of FIG. 9A.

FIGS. 9C and 9D show exemplary reconstructions for representative Ti/SiO₂ structures in the phantom, using HA density and Ti density.

FIG. 10A shows the dental phantom arrangement used for imaging a gold crown positioned against tooth material.

FIG. 10B shows a ray-traced projection image for the gold crown of the phantom at different angular orientations.

FIG. 10C shows, in an axial view, a reconstruction of the phantom tooth with gold crown using basic attenuation reconstruction.

FIG. 10D shows a reconstruction of the phantom tooth using density of an HA basis material.

FIG. 10E shows a reconstruction of the phantom tooth using density of titanium as basis material.

FIG. 11A shows a tooth with a metal crown.

FIG. 11B shows an x-ray projection image for the tooth of FIG. 11A.

FIG. 11C shows a reconstruction under HA density.

FIG. 11D shows a reconstruction using Ti density.

FIG. 12A shows a tooth reconstruction from an imaging phantom with a simulated gold-filled cavity embedded within HA material.

FIG. 12B shows a tooth reconstruction having a cavity of amalgam.

FIG. 12C shows a reconstruction of phantom dentition, generated using overall attenuation, having both a gold filling and an amalgam filling, along with gutta-percha filling and titanium posts.

FIG. 12D shows a reconstruction formed using HA basis density.

FIG. 12E shows a reconstruction formed using Ti basis density, isolating gold and Ti features from lower density structures.

FIG. 13A shows a photograph of a tooth having a metal filling and imaged as related to different basis materials using methods of the present disclosure.

FIG. 13B shows a view of a reconstructed image of the tooth, generated using overall attenuation.

FIG. 13C shows a reconstruction of the tooth bony tissue using density of an HA basis material.

FIG. 13D shows a reconstruction of the metal filling using density of titanium as basis material.

FIG. 14A shows a reconstruction using conventional attenuation imaging.

FIG. 14B shows a reconstruction for soft tissue visualization using soft tissue (ST) basis material.

FIG. 14C shows a reconstruction for soft tissue visualization using a mix of ST and HA densities.

FIG. 15A shows a reconstruction of a polyethylene block having holes filled with water, generated using lower-energy spectrum image content.

FIG. 15B shows a reconstruction for the polyethylene component, with water removed from the image content.

FIG. 15C shows a reconstruction for the water component, segmented from the polyethylene block.

FIG. 16A shows a projection image of a head phantom acquired using ST as a basis material.

FIG. 16B shows a projection image of the head phantom acquired using 50% ST-50% HA composite.

FIG. 17A shows an exemplary acquisition parameters setup screen.

FIG. 17B shows an exemplary material parameters setup screen according to an embodiment of the present disclosure.

FIGS. 18 and 19 show exemplary decomposition selection screens for choosing aspects of projection decomposition at a detailed level.

FIG. 20 shows views of multiple channels that can be applied to a single spectral scan.

FIG. 21 shows a composite user interface display allowing channel selection and different views of the acquired image content.

FIG. 22 shows an operator interface using a mouse or other pointer to scroll through data by channel number.

FIG. 23 shows an alternate method of user channel selection using an icon.

FIG. 24 shows how channel parameters selection can be used to provide improved segmentation.

FIG. 25 shows selection of specific channels enabling visualization, or removal. of particular materials in 2D or 3D views.

FIG. 26 shows virtual removal of sealant material that had been deposited upon the dentition.

FIG. 27 shows virtual removal of restorative material.

FIG. 28 shows an example of virtual tooth extraction.

FIG. 29 shows an example with virtual tooth extraction, as an aid to root canal treatment.

FIG. 30 shows virtual brace removal in orthodontic treatment.

FIG. 31 shows a display of separated or decomposed calcifications and soft tissue.

FIG. 32 shows displayed material separation, with highlighting of alveolar bone structures.

FIG. 33 is a diagram showing virtual removal of dentures and surgical guides in restorative dentistry.

FIG. 34 shows different 3D renderings by material for various types of anatomy.

FIG. 35 is an exemplary 3D rendering of intraoral features.

FIG. 36 is a diagram showing use of spectrally enhanced images in bone grafting for restoration and implant procedures.

FIG. 37 shows imaging for osseointegration assessment.

### DESCRIPTION OF EXEMPLARY EMBODIMENTS

The following is a detailed description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

In the context of the present disclosure, the terms “pixel” and “voxel” may be used interchangeably to describe an individual digital image data element, that is, a single value representing a measured image signal intensity. Conventionally an individual digital image data element is referred to as a voxel for 3-dimensional volume images and a pixel for 2-dimensional images. Volume images, such as those from CT or CBCT apparatus, are formed by obtaining multiple 2-D images of pixels, taken at different relative angles, then combining the image data to form corresponding 3-D voxels. For the purposes of the description herein, the terms voxel and pixel can generally be considered equivalent, describing an image elemental datum that is capable of having a range of numerical values. Voxels and pixels have the attributes of both spatial location and image data code value.

In the context of the present invention, the terms “digital sensor” and “digital detector” are considered to be equivalent. These describe the panel that obtains image data in a digital radiography system.

The term “highlighting” for a displayed feature has its conventional meaning as is understood to those skilled in the information and image display arts. In general, highlighting uses some form of localized display enhancement to attract the attention of the viewer. Highlighting a portion of an image, such as an individual organ, bone, or structure, or a path from one chamber to the next, for example, can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at higher sharpness or contrast.

A “conditioned” image is modified in some way by image processing software, such as to accentuate or suppress visibility of various imaged features according to material composition as described herein, for example. The term “technique” is a term of art in radiography and relates to exposure settings and adjustments for acquiring the x-ray image.

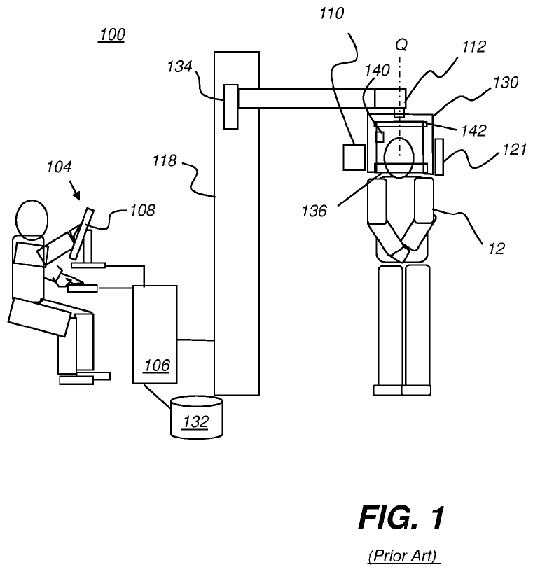

The schematic diagram of FIG. 1 shows an extraoral scanning imaging apparatus 100 that can be used for 2-D or 3-D imaging of teeth and other intraoral features. In 2-D imaging, a single image can be acquired, or multiple 2-D images can be acquired, such as in panoramic imaging, in which a succession of two or more 2-D images is obtained and images of adjacent content are arranged to form a larger image. For 3-D imaging modes, such as tomography, computed tomography volume imaging, or cone beam computed tomography (CBCT) imaging, multiple 2-D images are acquired in series and used to reconstruct features for dental, ENT, and related head imaging applications.

In the FIG. 1 schematic, a rotatable mount 130 is provided on a column 118, preferably adjustable in height to suit the size of patient 12. Mount 130 maintains x-ray source 110 and a radiation sensor 121 on opposite sides of the head of patient 12 and, optionally, rotates to orbit source 110 and sensor 121 in a scan pattern about the head. Mount 130 rotates about an axis Q that corresponds to a central portion of the patient's head, so that its attached components orbit about the head. Sensor 121, a photon-counting sensor according to an embodiment of the present invention, is coupled to mount 130, opposite x-ray source 110 that emits a radiation pattern suitable for 2-D imaging, for tomosynthesis imaging, or for CT or CBCT volume imaging. An optional head support 136, such as a chin rest or bite element, provides stabilization of the patient's head during image acquisition. A computer 106 has an operator interface 104 and a display 108 for accepting operator commands and for display of volume images obtained by imaging apparatus 100. Computer 106 is in signal communication with sensor 121 for obtaining image data and provides signals for control of source 110 and, optionally, for control of a rotational actuator 112 for mount 130 components. One or more height sensors 134 is also sensed by computer 106 in order to obtain an initial height setting and to track relative vertical displacement of the sensor 121 relative to the patient's head during the helical scan. Computer 106 is also in signal communication with a memory 132 for storing image data. An optional alignment apparatus 140 is provided to assist in proper alignment of the patient's head for the imaging process. Alignment apparatus 140 can include a laser that provides one or more line references for head positioning according to an embodiment of the present invention. In alternate embodiments, alignment apparatus 140 includes a visible light beam or other marker, or a mechanical or other positioning apparatus. Imaging apparatus 100 may also have the capability for panoramic or cephalometric imaging using x-ray source 110 and sensor 121 or other imaging sensor.

There can be a number of variable scan patterns according to the type of imaging that is required. Tomosynthesis, for example, typically uses a scan that is less than 180 degrees about the patient. CBCT scanning may require a helical scan pattern with one or more revolutions about the patient's head. An optional adjustment mechanism 142 is provided for adjusting the source-to-image (SID) distance between the x-ray source 110 and sensor 121 to suit the scan pattern for different patients or types of imaging.

As noted previously in the background section, conventional radiography provides image content based on attenuation coefficients along each path of the x-ray beam. In an image having a number of different materials, such as a dental x-ray or cephalometric image, the conventional x-ray provides very limited information related to materials in the imaging path. The integrating x-ray sensors that are conventionally used are spatially digitized and provide an analog output that represents the accumulated charge received for each pixel during the exposure interval. This imaging method is typically subject to noise.

Spectral x-ray imaging uses an alternate approach to image capture, in which radiation over a relatively broad spectral range, or of two or more energy ranges, is directed through the subject. Each energy range is detected over a pixel or other region, and the combination of energy received can be used to characterize the material composition of the subject over that region.

Notable types of spectral, polychromatic, or “color” x-ray imaging include:
(i) dual-energy imaging, in which two x-ray images of the same subject anatomy are acquired in rapid sequence, each at a different energy level, and the images merged; and
(ii) photon counting, in which incoming x-ray photons above and below different threshold values are counted in order to provide the image data for each pixel.

Embodiments of the present disclosure can use either dual-energy or photon-counting x-ray apparatus for dental imaging. Advantageously, spectral x-ray imaging yields information beyond the attenuation coefficient for the composite subject that is imaged, including information that is indicative of the actual density of the imaged material (in units of

$$\frac{g}{{cm}^{3}} ,$$

for example).

### Photon-Counting Advantages

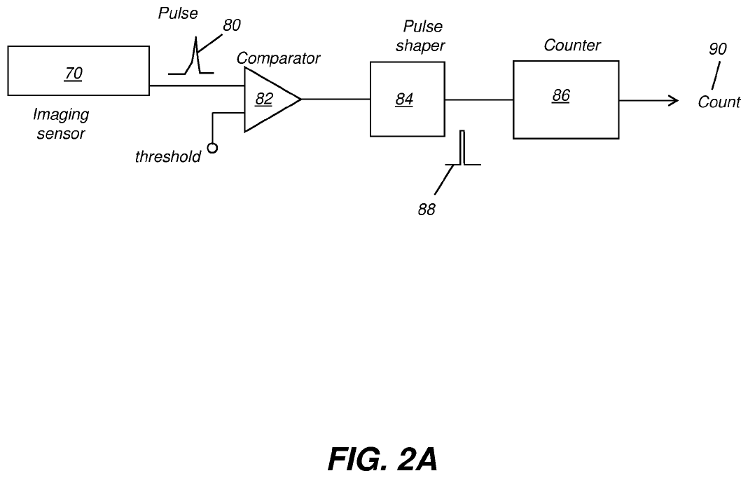

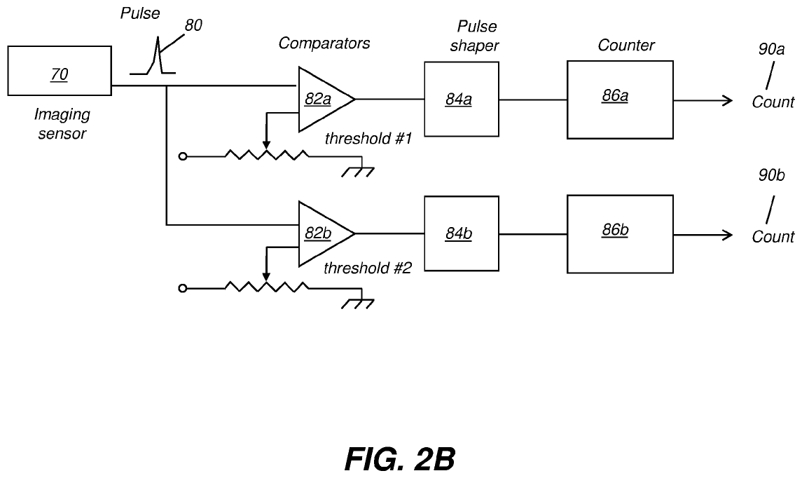

Spectral x-ray using a photon counting detector array provides for low cost and low dose color x-ray imaging. Advantageously, photon counting has high immunity to noise, provided that pulse strength exceeds background noise levels. FIG. 2A shows the photon-counting sequence in schematic form. An incoming photon generates a pulse 80 at a given energy level. For each array position or “pixel”, the pulse 80 energy is compared against a threshold value at a comparator 82 and shaped in a pulse shaper 84 to form a shaped pulse 88. A counter 86 then records the pulse event and provides a digital output, a pulse count value 90. A separate pulse count value 90 is obtained for each pixel element in the imaging sensor 70 array. The threshold value can be adjustable or selectable from a range of values, depending on the photon energies of interest. Photon counting x-ray detectors provide suitable performance at a low signal level, and therefore allow reducing the x-ray dose given to a patient.

A further advantage of pulse counting relates to its capability to count pulses 80 at multiple threshold values at each pixel location. Referring to the schematic diagram of FIG. 2B, two comparators 82a and 82b are shown for measuring pulse energy. In this particular configuration, a comparator 82a, a pulse shaper 84a, and a counter 86a provide a count 90a value for all pulses received, at that pixel location, at energies above a first threshold; similarly, a comparator 82b, a pulse shaper 84b, and a counter 86b account for only pulse energies above a higher, second threshold and provide a count 90b accordingly. Simple subtraction then identifies the different power levels achieved for each pulse. It can be appreciated that more than two threshold levels can be measured at a pixel, using a corresponding arrangement of comparator circuitry, allowing pulse counts corresponding to photon energies at energy ranges defined by any of a number of threshold values. In addition, thresholds can be selectable with some systems, such as being adjustable to fine-tune the response of imaging sensor 70 to various photon energy levels. Thus, for example, an operator can use a set of preset thresholds for differentiating softer from denser tissue in the conditioned image that is finally generated.

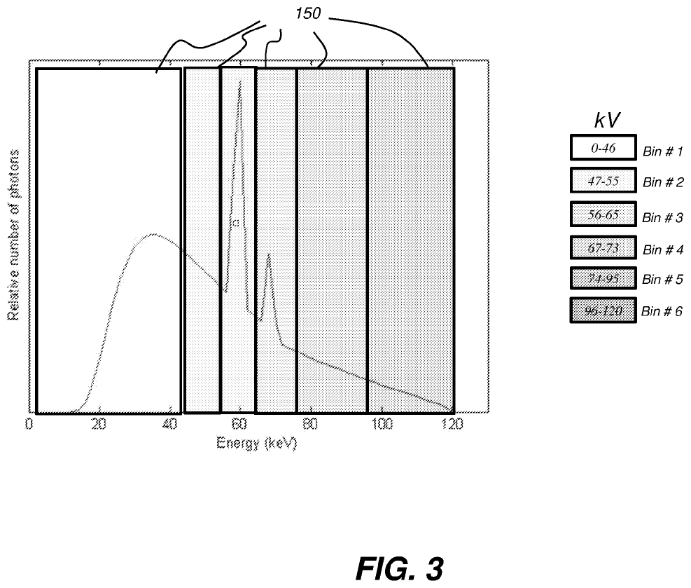

The graph of FIG. 3 shows an arrangement of six energy bins 150 defined over the energy (keV) domain. The boundaries and width of each bin 150 are selected in order to facilitate materials characterization in response to incident x-ray energy. Embodiments of the present disclosure can provide one or more default bin distributions, selectable by the end-user according to the type of exam, or according to the condition of the patient. Embodiments of the present disclosure can alternately use information from imaging results to specify or to adjust threshold kV values that define the bin 150 boundaries.

In addition to setting minimum thresholds, embodiments of the present invention can also provide the option of using upper or maximum thresholds for enumerating photons to measure photon energy. This capability can be used for a number of functions, including reducing the generation of excessive noise signals such as from metal artifacts or x-rays passing directly through the direct detection material.

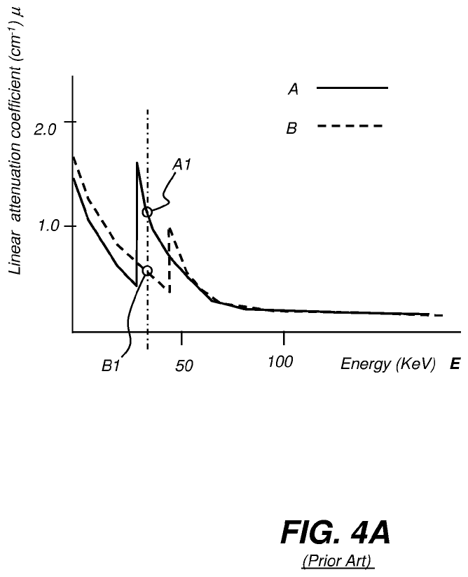

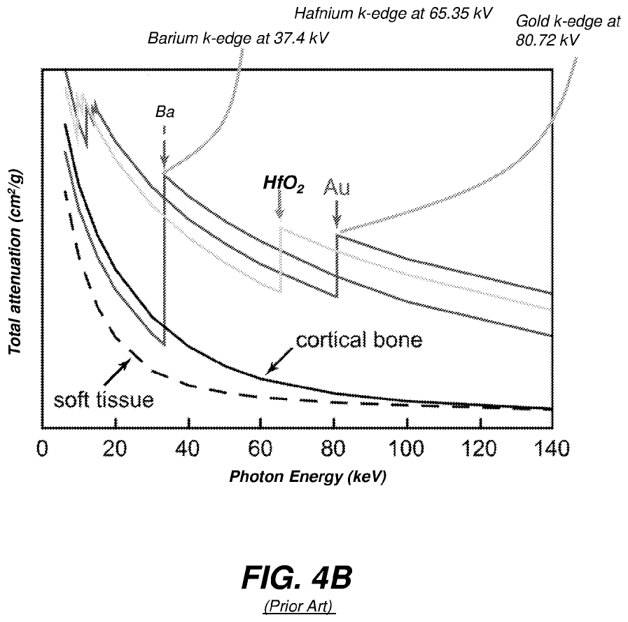

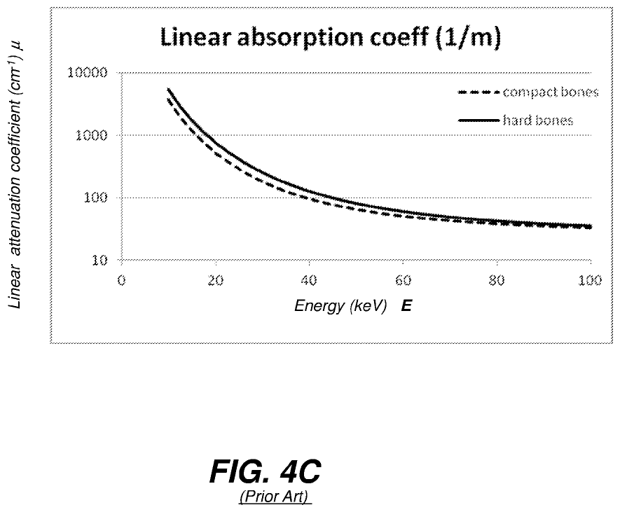

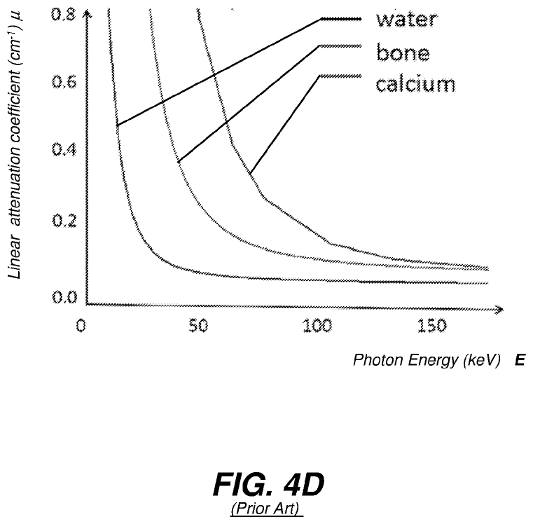

The capability to distinguish and count photons at different energy thresholds, as described with reference to FIG. 2B, allows the sensor to differentiate between energy levels obtained from irradiating the subject and provides added dimension to the image data that is provided as a result of each exposure. This capability, described as multi-spectral or “color” x-ray imaging, enables information to be obtained about the material composition of a subject pixel. As shown for typical metals in the simplified graph of FIG. 4A, two materials A and B have different coefficients of attenuation μ that vary with the level of radiation energy, shown as exposure E. At a given exposure value, shown as a KeV value, material A attenuates a photon with an energy absorption that corresponds to material A, as shown at value A1. Similarly, radiation impinging on material B attenuates a photon with an energy that corresponds to material B, as shown at value B1. This variable characteristic of an imaged material can be considered a type of spectral “signature” for the material, used as described subsequently in this disclosure. Where photons of these different energy values can be differentiated from each other, it is possible to identify one or both materials in the same pixel or voxel image element of the obtained image. This same basic behavior in response to radiation also allows some measure of capability to differentiate tissue and material types.

By way of example, the graph of FIG. 4C shows relative coefficients of attenuation for two different bone densities. The graph of FIG. 4D shows curves of relative coefficients of attenuation for water, bone, and calcium. Over a range of keV values, the steepness of the curve is an indicator of higher attenuation and thus of material content. As FIGS. 4C and 4D suggest, different linear absorption characteristics allow differentiation between various types of tissue, such as between bone types, as well as between different types of materials.

As one significant difference from conventional large-area image detection, the photon-counting architecture generally uses an image detector of reduced size, typically requiring a scanning sequence even where only a 2-D image is obtained. For volumetric or 3-D imaging, such as in the sequence needed for CT or for cone-beam CT (CBCT) imaging, it may be necessary not only to scan within the same plane, but additionally to provide a 3-dimensional helical scan.

### Exemplary Applications

The enhanced information available using spectral radiographic imaging allows capabilities for image conditioning such as virtual material removal, as described herein, as well as improved results for materials identification and mapping, more precise segmentation, improved 3D reconstruction, and more accurate materials decomposition data, and can be used to support a number of dental procedures and conditions, including the following:
Root canal geometry assessment, allowing visualization with removal of the root canal filing from image content, allowing visibility of inner surfaces of the root canal;
Osseointegration assessment of an implant, with visualization of bone structure distinguished from integrated titanium features;
Visualization of tooth structure with crown removal for supporting endodontic treatment;
Tooth decay assessment, with visibility of tooth structure separate from filling;
Soft-tissue only images for root canal and pulp chamber assessment;
Soft-tissue only images for gum assessment;
Cephalometric analysis with soft-tissue and bone/tooth only images.
Visualization of intraoral features with braces or other prosthetic devices removed;
Bone structure assessment, including the ability to characterize bone density and other features of overall bone health;
Graft volume assessment;
Improved identification of materials used in treatment of various types, including metals, ceramics, plastics, and sealants, for example, as well as accurate information on spatial positioning of various features for use in generating and updating patient chart data;
Highlighting of materials used within the mouth according to type;
3D reconstruction for visualization of features according to material type;
Implant interface assessment; and
Visualization in 2D or in 3D according to materials profile or “channel” setup.
Measuring the dimensions of an implant or the part of the bone into which the implant is inserted.
Measuring the dimension of a filling material or the tissue that is filled or planned to be filled.

### Varied Image Types

Types of radiographic images that can be obtained and used for improved materials characterization using embodiments of the present disclosure can include:
Scout images acquired with a 3D dental scanner;
Panoramic images;
Tomosynthesis images acquired by moving a source (or by using multiple source focal spots) with a fixed detector;
Intraoral images with the detector held in the patient's mouth,
Intraoral tomosynthesis images;
A portion of a reconstructed volume, such as an axial, coronal, or sagittal view or a slice of any orientation from a reconstructed volume of images from a 3D scan; and
Cephalometric images.

### Applying ALARA Principles

Embodiments of the present disclosure can help to support principles of “as low as reasonably achievable” (ALARA) dose for radiation imaging. The use of low-dose scout images, for example, can provide significant information on intraoral features and material composition for fillings and prostheses, so that dose levels for a full imaging sequence can be kept as low as possible.

### Profile or “Channel” Setup

Embodiments of the present disclosure take advantage of the spectral “signature” that is available for the different natural materials, as well as for processed or prepared man-made or “fabricated” materials used for replacement, adjustment, repair, or restoration of features within the mouth that can be imaged using radiographic tools. As was shown briefly in the example graphs of FIGS. 4A-4D, different materials exhibit different levels of attenuation to x-ray energy at various wavelengths or, alternately considered, at various photon energy levels. Thus, considering the graphs in FIG. 4A for example, it is possible to determine material composition of a subject by analyzing its attenuation over a range of wavelengths, and to distinguish subject materials “A” and “B”, such as distinguishing one metal from another, one type of living tissue from another, or one composite filling material type from another, for example.

With spectral x-ray exposure, variable attenuation over the spectrum, or even over particular portions of the spectral domain, is characteristic of each imaged material, whether naturally occurring and “native” to the oral anatomy or fabricated in some way, treated, shaped and configured from suitable materials and positioned, permanently or temporarily, within the mouth. Embodiments of the present disclosure take advantage of the added dimensions of spectral imaging by enabling the use of channels. A channel can be considered a predetermined domain mapping that is optimized to collect and measure photon energy, over a sufficient number of energy levels, in order to characterize material content of the imaged subject. From a related aspect, a channel can relate to a density profile, where spectral response to one or more density values is characteristic of a basis material. Channel mapping can allow the viewer to distinguish materials, both natural and synthetic, from each other. According to an embodiment, each channel identifies a particular combination of one or more basis materials used for material decomposition processing of the resulting image.

Considering the simplified attenuation coefficients graph for materials A and B in FIG. 4A, it can be appreciated that collecting photon data at particular threshold values can provide sufficient information for characterizing and distinguishing materials A from B in a given object. Using this capability, a channel can be set up that identifies specific energy “bins”, each bin defining a specific energy range for collecting photon data; this channel also provides the needed data for materials decomposition processing, allowing the material to be identified, as well as used for display highlighting and reconstruction.

The channel can alternately be considered as an arrangement of basis materials or energy bands used as reference factors for material decomposition. A channel provides a vehicle for interpreting relative photon energy levels as they relate to particular materials in the imaged subject. As is known to those skilled in photon-counting technology, material decomposition can be effected by analyzing the energy information obtained at different photon energy levels, as acquired using the channel features described herein.

Thus, channel setup defines an arrangement of spectral domains sufficient for characterizing one or more materials in the acquired image content according to photon energy levels and quantization settings. How these channels can be used to advantage is described in more detail subsequently in the present disclosure.

Steps for channel setup include:
(i) define a number of energy bins for photons detected when irradiating the material;
(ii) relate the defined arrangement of energy bins to a set of basis materials that serve as benchmarks for imaged materials;
(iii) relate the basis materials to actual materials found in the intraoral environment, whether naturally occurring (bone, tooth, or other tissue) or non-native to the intraoral environment, such as man-made or fabricated materials and features;
(iv) store the resultant values obtained from steps (i)-(iii) as decomposition factors for processing intraoral radiographic image content.

According to an embodiment of the present disclosure, channel setup can have a number of initial settings assigned at manufacture; these settings can be modified by the practitioner at a particular site in order to make the channel more usable for particular patients or practice requirements.

Spectral radiography can also take advantage of k-edge materials response for various materials subjected to incident radiation, as shown in FIGS. 4A and 4B. Elements such as barium, hafnium, and gold exhibit a distinctive and abrupt k-edge response at characteristic radiation energies. The k-edge for barium is at 34.7 kV; for hafnium at 65.35 kV; for gold at 80.72 kV. This response can be helpful in identifying presence of the material, such as barium, hafnium, or gold in a dental coating or in sealants or other materials, for example.

### Materials of Interest

In supporting the above-listed applications and other dental applications where visualization according to material type can be useful, there are a number of different materials of particular interest for dental radiography, both native to the anatomy and added temporarily or permanently as fabricated, including the following: soft tissue, bone, tooth enamel, gutta-percha, sealants, coatings, and various polymers, compositions, ceramics, and metals. Each of these types of materials in this set has a corresponding spectral range, typically expressed in terms of KeV, that is best suited for characterization in radiographic imaging, both for 2-D radiography and for 2-D projection image acquisition that is used for subsequent 3-D reconstruction and for materials decomposition processing that provides information on the material composition of various features. Embodiments of the present disclosure are directed to particular materials, typically within the subset of materials used in dental and medical treatment, both for acquisition and for subsequent processing and visualization. This includes, but would not be limited to, materials listed in Table A.

As a practical approach to characterizing the imaged materials that are common to the intraoral environment, embodiments of the present disclosure can employ a set of two or more basis materials as a reference set for determining energy levels for x-ray exposure and as factors for performing the processing required for material decomposition processing based on the energy detected from exposure. Basis materials provide well-known references against which particular measurements of an imaged sample can be assessed. In practice, energy binning and corresponding quantization corresponds to basis materials selection in the approach used in embodiments of the present disclosure.

Unlike other forms of intraoral imaging, an embodiment of the present disclosure facilitates detection and identification of sealants, including glass ionomer (acid-base reaction), resin-modified glass ionomers, polyacid-modified resins (compomer), and composite resins. Spectral imaging can be used to distinguish non-eugenol type sealants, including calcium hydroxide-based, resin-based, solvent-based, nogenol-based, silicon-based, calcium phosphate-based, urethane methacrylate-based, and MTA-based sealers from eugenol sealers. Some types of eugenol sealers include sealers containing zinc-oxide eugenol, Kerr-Pulp canal sealer from Kerr Corporation, Brea CA; Grossman's sealer, TubliSeal™ from Kerr Corporation (SyberonEndo), Brea, CA; Endofill Root Canal Filling Material from Dubai Medical Equipment, Sharjah, UAE; and Zical® Root Canal Sealant from Prevest Denpro, (Bahadurgarh, Haryana, India).

TABLE A
Materials of Interest

| | | Density | |
|---|---|---|---|
| Material | Composition | (g/cm³) | Uses |
| ICRU 4-Element | 10.1% H | 1.0 | Component of |
| Soft-Tissue (ST) | 11.1% C | | Tooth, Bone, Tooth |
| | 2.6% N | | Pulp Chamber, Root |
| | 76.2% O | | Canal |
| Hydroxyapatite (HA) | Ca₅(PO₄)₃(OH) | 2.98 | Component of |
| | | | Tooth, Bone |
| Enamel | 95% HA, 5% ST | 2.88 | Tooth Crown |
| Dentin | 45% HA, 55% ST | 1.89 | Tooth Crown, Root |
| Stainless Steel | C: 03 to .15% | 7.85 to | Orthodontics/ |
| (Ferritic, Martensitic, | Cr: 12-29% | 8.06 | Crowns/Fillings |
| Austenitic, Duplex, | Ni: 0.75 to 12% | | |
| precipitation- | Fe: >15% | | |
| hardening) | Traces: Mo, Mn, | | |
| | S, P, Si | | |
| Titanium | Ti | 4.54 | Implant Post |
| Amalgam | 50% Hg | 11.07 | Cavity Filling |
| | 30% Ag | | |
| | 10% Cu | | |
| | 10% Sn | | |
| Gold | Au | 19.32 | Cavity Filling, |
| | | | Crown |
| Gutta-Percha | 20% C₅H₈ | 2.21 | Pulp Chamber, Root |
| | 66% ZnO | | Canal Filling |
| | 11% BaSO₄ | | |
| | 3% H₂O | | |
| Silicone Dioxide | SiO₂ | 2.32 | Crown |
| (Ceramic) | | | |
| Zirconium Dioxide | ZrO₂ | 5.68 | Crown |

Denture materials that can be detected using the techniques described herein can include vulcanite, nitrocellulose, Chrome cobalt, porcelain, and various synthetic resins.

Typical restorative materials can include gold, porcelain, composite resins, amalgam, ceramics, and zirconia, for example.

Gutta percha is a plant-derived polymer product that is fabricated by an extraction and refinement process that separates it from other materials.

### How Basis Density is Used

Spectral x-ray imaging itself is generally not useful for accurate differentiation of different types of soft tissue and fluids; this is due to the relatively close atomic number values for constituent atoms that are predominant in these materials, such as carbon (6), nitrogen (7), and oxygen (8). The use of basis materials helps to compensate for this difficulty in material decomposition processing. The basis material provides a useful reference for relating the signal acquired from attenuation of the measured energy (that is, the acquired photon count) to the relative density of a material. Decomposition using different soft-tissue-like basis materials helps to enhance the usefulness of signal content for distinguishing various types of soft tissue from each other. The resulting images can be noisy; however, noise reduction methods allow some measure of correction to compensate for these effects.

Basis material images can be used to auto-detect regions of a specific soft-tissue type. 2-D imaging modalities particularly benefit, since overlapping bone and tooth content can be readily removed from the image.

Table B lists exemplary basis materials for typical applications and corresponding materials. Decomposition processing is executed according to these or other suitable basis materials, each basis material providing a useful reference for relating the acquired photon energy levels and photon count to a particular material in the image.

TABLE B
Example Basis Materials and Reconstruction
Content by Application

| | | Basis |
|---|---|---|
| Application | Reconstruction content | material |
| Metal artifact reduction | All tissue | HA |
| | Metal | Ti |
| Metal filling separate from | All tissue | HA |
| cavity | Metal | Ti |
| Composite filling separate | Pulp chamber, root canal, | ST |
| from cavity | dentin | |
| | Enamel, ceramic | HA |
| Gutta-percha pulp chamber | All tissue | HA |
| and root canal removal | Gutta-percha | Ti |
| Ceramic crown separate from | Pulp chamber, root canal, | ST |
| tooth structure | dentin | |
| | Enamel, ceramic | HA |
| Metal crown separate from | All tissue | HA |
| tooth structure | Metal | Ti |
| Ti implant from surrounding | All tissue | HA |
| tooth, bone | Metal | Ti |
| Panoramic and cephalometric | Soft tissue, pulp chamber, | ST |
| images, soft tissue and | root canal | |
| bone/tooth only | Bone, enamel, dentin, metal, | 50% ST |
| | ceramic, gutta-percha | 50% HA |

### Imaging Sequence

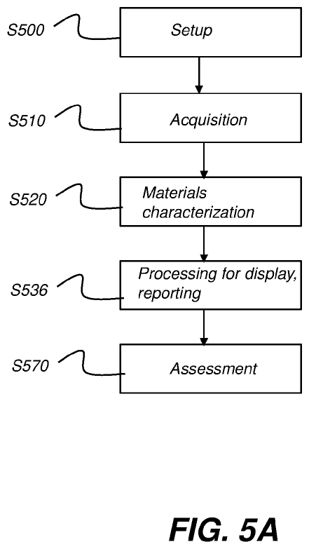

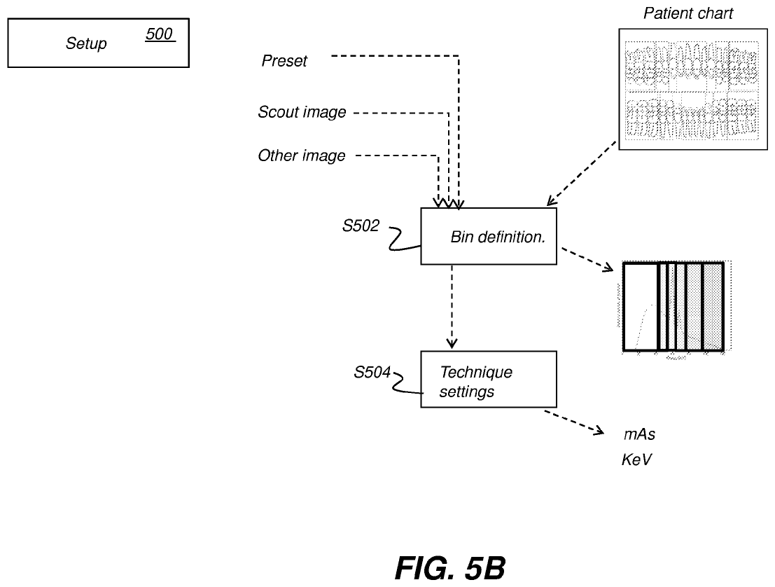

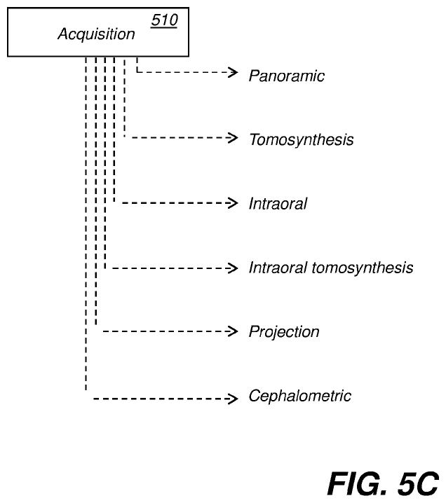

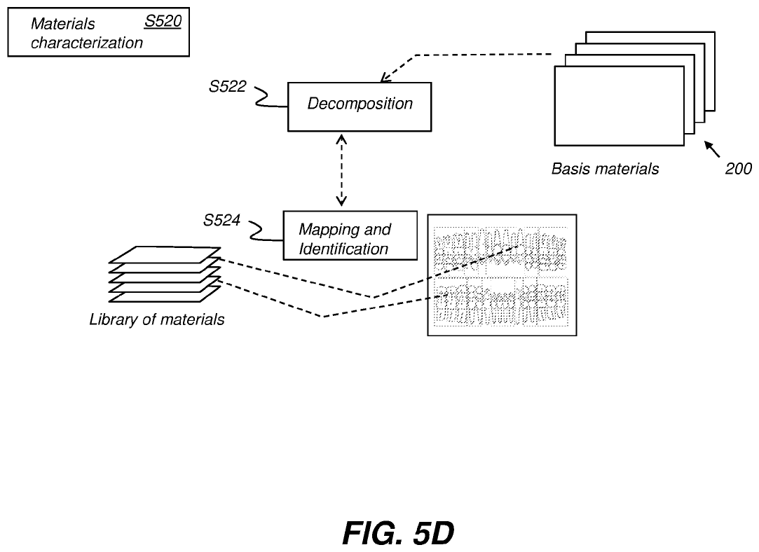

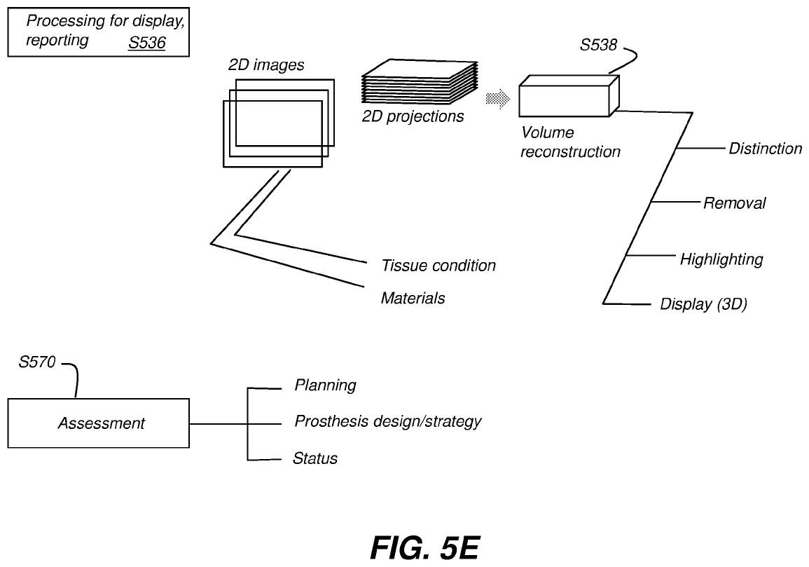

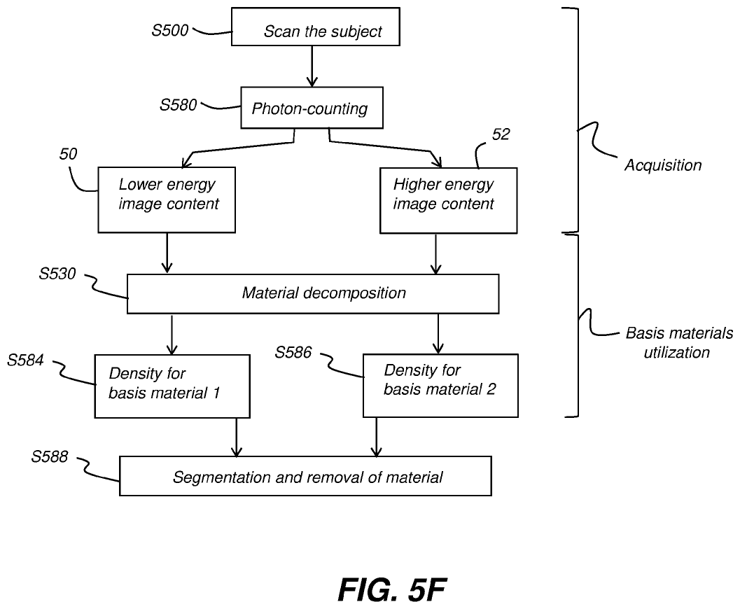

The flow diagrams of FIG. 5A through 5F show a sequence of steps for acquisition and processing of image content for material and features characterization according to an embodiment of the present disclosure. The example process shown in FIG. 5A shows an overview of the imaging process according to an embodiment of the present disclosure.

Referring to FIG. 5A, a setup step S500 sets up the energy levels for photon counting, including defining the number of bins 150 (FIG. 3) and the energy domain associated with each bin, as determined by energy threshold value settings. Step S500 can also include defining the radiography technique settings for obtaining image content.

The object that is imaged by the radiographic system (alternatively termed the “subject” for imaging) is exposed and corresponding image data collected in an acquisition step S510. A materials characterization step S520 then provides decomposition computing for the acquired image data. Decomposition logic can employ a variable set of basis materials, for example. A processing step S536 processes the data for 2D or 3D display, according to the acquired data content. As assessment step S570 then allows manual or computer-assisted evaluation of patient condition and treatment strategy for the processed image content.

### Bin Setup

The logic flow sequence of FIG. 5B shows setup step S500 in more detail. Bin definition step S502 sets the threshold value settings that define energy levels and resolution for the photon counting detector. The threshold settings can be provided from stored preset values, with sets of threshold energy value settings arranged beforehand and indexed according to factors such as patient age and personal condition, size, medical and dental history, treatment indications, and other factors.

The patient chart can be used as input to bin definition calculations, that is, for energy threshold value settings corresponding to bin boundaries. The existence of a crown or of post-and-core fixtures within the patient's mount can help to determine how best to measure the photon energy from the exposure process.

Alternately, an initial low-dose scout image of the subject can be obtained and used to generate a coarse determination of material composition for intraoral features, sufficiently accurate for defining threshold value settings for the energy bins, as well as for updating patient chart data.

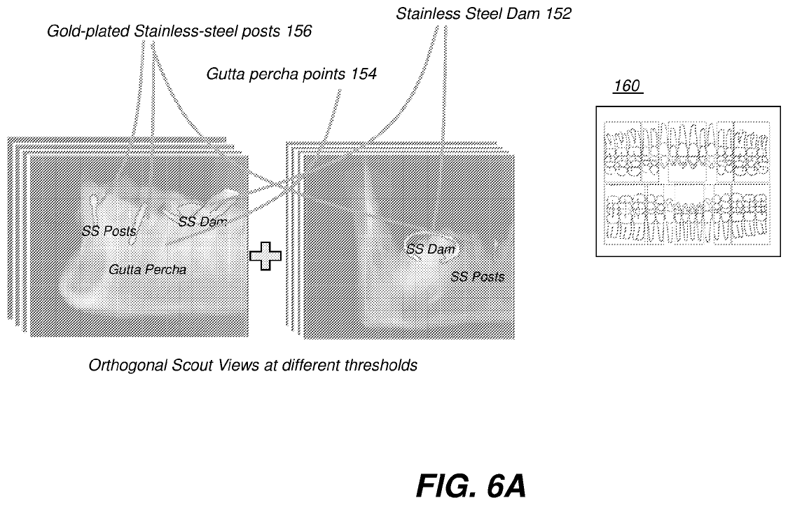

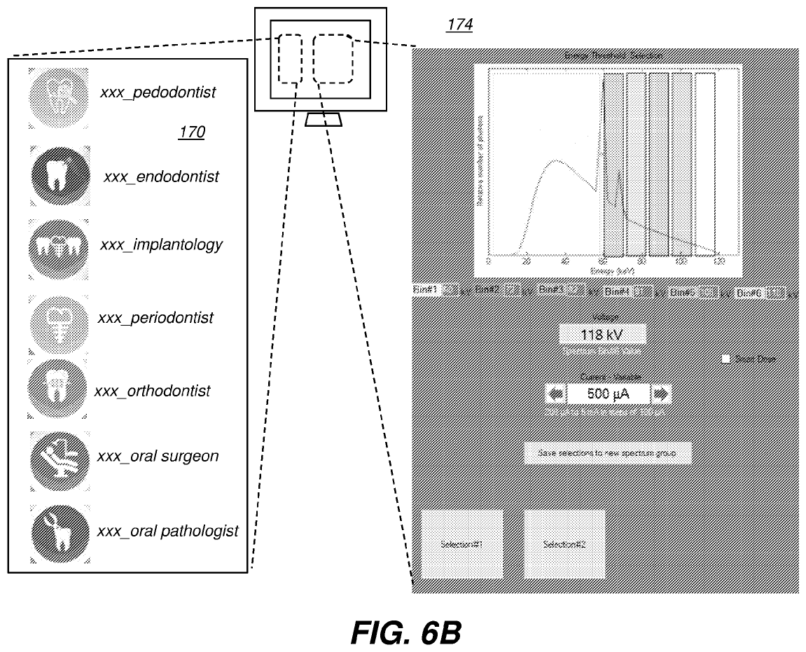

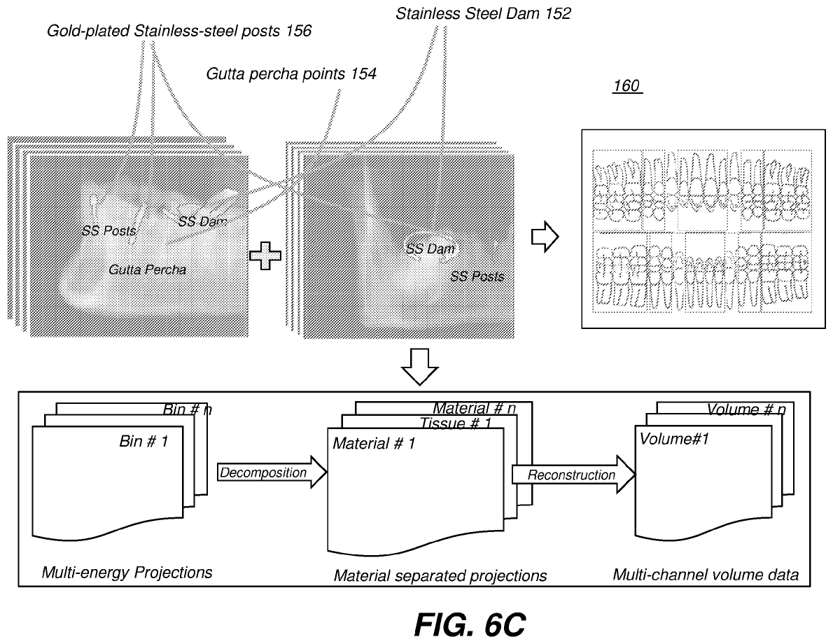

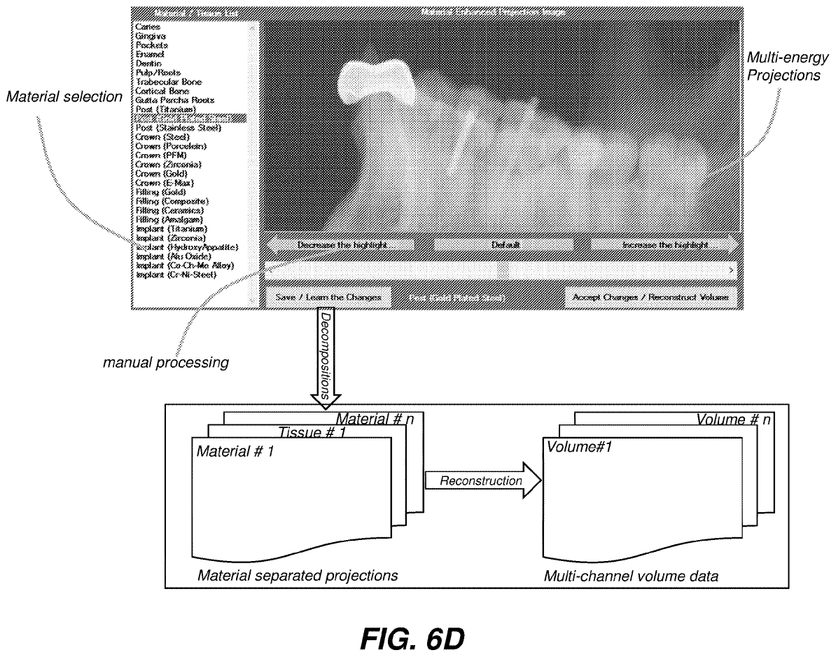

FIG. 6A shows, by way of example, the use of scout image content for populating a dental chart 160 for the patient, in addition to defining or adjusting the bin 150 thresholds. A stainless steel dam 152, gutta percha points 154, and metal posts 156 can be detected by the scout image and chart 160 suitably enhanced or annotated. In the FIG. 6A example, dental chart 160 is labeled by the system to denote positions of detected materials from the scout image.

FIG. 6B shows other methods that can be used for editing bin definition energy thresholds on a user interface associated with the x-ray imaging system. A set of selection buttons 170 enable selection of default bin threshold settings according to a specialization type or treatment. A manual adjustment interface 174 can alternately be used, allowing a user to enter values that set up or adjust threshold values that define each bin. This facility can allow bins to be added or combined as needed, also including technique settings, for example.

In addition to the scout image, other image results could also be used, such as recent images obtained from the same patient, for example. An optional technique settings step S504 can set or adjust settings for exposure current and voltage potential to be used.

### Image Acquisition

Following bin setup, acquisition step S510, as shown in the diagram of FIG. 5C, then executes, generating one or more images of the subject patient. Various types of images can be obtained, depending on the imaging apparatus used and the patient need, including static x-ray images, panoramic images, tomosynthesis projection images, intraoral images, intraoral projection images, and cephalometric images, for example.

### Materials Decomposition Processing

The image data includes attenuation information that can be decomposed in a process that determines the types of naturally occurring or fabricated materials encountered by the exposure radiation of the subject. Materials characterization step S520, as shown in FIG. 5D, can then perform the needed processing for materials identification in a decomposition step S522, with mapping and reporting provided in a mapping and identification step S524. One or more basis materials 200 are defined for the image processing system, as representative or “benchmark” materials for modeling the attenuation response of actual materials in the subject. For example, water can serve as a basis material for many types of bodily fluid, and as a basis material for many types of soft tissue (ST) features, since soft tissue has negligible mineral content; hydroxyapatite (HA) can serve as a basis material for tooth or bone features. In many cases, values from two or more basis materials are used to identify a particular material.

For the purpose of describing exemplary embodiments herein, bone-like tissue mineral content (e.g., bone-like tissue of varying mineral density) is preferably modeled by the mineral hydroxyapatite (HA), which serves as a useful basis material 200. However, definition of bone-like tissue mineral content may vary. The density ρ at every point in the object has a density value that is a summation of the density of soft tissue (ST) and HA, respectively ρ_{ST} and ρ_{SA}, multiplied by a weighting factor as shown in equation (1):

$$ρ = {f}_{ST} {ρ}_{ST} + {f}_{HA} {ρ}_{HA} \qquad ( 1 )$$

wherein weighting factors f_{ST} and f_{HA} are fractional values for soft tissue and hydroxyapatite, respectively.

Using the model expressed in equation (1), the X-ray attenuation coefficient μ at every point in the exposed object is given by equation (2):

$$μ = ρ ( {f}_{ST} {\hat{μ}}_{ST} + {f}_{HA} {\hat{μ}}_{HA} ) \qquad ( 2 )$$

wherein \hat{μ}_{ST} and \hat{μ}_{HA} are the mass attenuation coefficients of the two basis materials ST and HA, respectively.

HA and ST are used in this example as two representative basis materials 200. It can be appreciated that any number of additional basis materials can be utilized in order to provide enhanced resolution for the materials decomposition process. A set or “library” of materials can be identified using the corresponding computation of basis materials provided by the materials characterization process in step S520.

Mapping and identification of materials within the patient's mouth can be performed in an automated manner, providing the practitioner with a populated dental chart. This capability can provide a chart for a new patient or can be used to update an existing dental chart, for example.

### Processing for Display and Reporting

Processing step S536, as shown in more detail in FIG. 5E, works with the acquired 2D images in order to provide further analysis for display. For 3D volume images, the 2D images are projection images, used to generate a volume image in a reconstruction process S538. Processing of the reconstruction can include further steps for distinguishing materials and for highlighting areas of the image where various materials of interest may be present. Improved segmentation can allow 3D visibility according to material, so that images can be provided that include or exclude only a desired set of materials, for example.

An assessment step S570 can provide additional display and analysis tools to assist in identifying patient condition and in treatment planning.

### Example Workflow for Materials Removal

FIG. 5F shows exemplary logic flow for a materials removal process in radiographic scanning. This process uses spectral imaging to characterize material content of the imaged subject according to attenuation data over two energy regions, obtained in a scanning step S500, using photon counting S580. A single threshold value is used to define a lower energy bin 50 for acquiring photons having energy beneath the threshold value and a higher energy bin 52 for photons at energies above the threshold value. Photon attenuation at two or more different energy levels can then be used to characterize the material content of each portion of the subject.

Photon energy acquisition itself can involve scanning the image by applying x-ray energy over a suitable spectral range that includes both lower and higher energy values. Other methods for scanning can include alternating lower and higher energy values within the same scan, or applying different energy in separate, consecutive scans. However, the photon-counting method is advantaged since it allows collection of spectral data in a single scan of the intraoral subject by applying exposure energy distributed over a broad spectral range and detecting the energy attenuation according to the relative energy level measured. While two energy levels are shown in the FIG. 5F sequence, photon-counting detectors may allow the use of multiple thresholds, allowing additional bins to be used for measuring photon data at additional energy levels.

The next part of the sequence relates to the selected basis materials and processing the acquired image content according to the basis materials. A material characterization step S520 employs the photon data in order to determine material composition, according to the acquired image content.

In the example of FIG. 5F, two basis materials are defined for mapping the corresponding attenuation data. A density computation step S584 obtains density information for a first basis material. A density computation step S586 obtains density for a second basis material. A material removal step S588 then performs segmentation and visualization or removal of material in the acquired or reconstructed image.

By “removal” is meant the masking or suppression of specific image data content that relates to a particular material or group of materials. A removal process identifies individual pixels, for example, that appear to represent a material of interest. Values for these pixels are adjusted to normalize the pixels to surrounding content, so that features formed of the material of interest are no longer visible or are sufficiently suppressed to allow visibility of surrounding features. This can be equivalent to varying image pixel or voxel opacity, for example, adjusting the opacity to very low values or to zero for transparency.

The overall sequence of FIG. 5A admits a number of alternatives that are suited to imaging various types of materials in acquiring both 2D and 3D images. In scanning step S500, the operator can select two or more appropriate energy levels for materials identification, based in the imaging type and on the materials of interest. For example, the lower energy value used for bin 50 can be optimized for soft tissue imaging. The higher energy value used for bin 52 can be optimized for bone.

Following acquisition, image processing in FIG. 5F proceeds using two basis materials selected for the particular imaging application. The two basis materials are typically selected from the set of water, soft tissue, bone, tooth enamel, gutta-percha, and various ceramics and metals, as noted previously. One selected basis material has lower attenuation coefficient and lower density. Processing for the lower density basis material can distinguish, from materials of higher attenuation, the selected material and any material of relatively lower density than the selected material.

The other selected basis material has higher attenuation coefficient and higher density. Processing for the higher density basis material can distinguish the selected material, and any material of relatively higher attenuation than the selected material, from lower density materials.

The FIG. 5A-5F process works most effectively when the practitioner has at least some fore-knowledge of the different materials that are present in the mouth being examined. This information helps to condition various aspects of the image acquisition process, including selection of basis materials and corresponding energy levels, for example.

The sequence of FIG. 5A-5F has been shown and described for dual energy acquisition. Scanning and acquisition apply lower energy and higher energy thresholds for the received photon energy, so that all photons related to materials below the lower threshold contribute primarily to the lower density material image content; all photons corresponding to material above the upper threshold contribute primarily to the higher density material image content. A similar sequence applies for photon-counting approaches that use three, four, or more energy levels for characterizing intraoral features of various materials. The use of additional energy levels can enable higher accuracy as well as differentiation of more types of material. Each energy level used has a corresponding basis material.

Distinctions can be made between various materials according to their relationship to the basis materials. Subsequent examples show the results of processing the acquired image data according to different sets of basis materials.

### 3D Volume Reconstruction

3D volume reconstruction can be performed in either of two ways for material removal. Using one approach, material segmentation and/or removal is applied to the acquired 2D projection images, prior to reconstruction. The processed 2D projection images can then be used to form a reconstruction for a selected material type. In an alternative approach, volume reconstruction can be performed first, using photon attenuation information for all material types; following this, decomposition of the 3D volume can be executed using identification according to the basis materials.

It can be appreciated that the combination of basis materials used for image processing can be changed following image acquisition, as was shown in the example of FIG. 5F. Thus, for the same image content, one subset of basis materials can be used for optimizing image acquisition; a different subset of basis materials can then be used for materials decomposition processing. This allows the flexibility to acquire and process image data under variable conditions, allowing different views and uses of the acquired image content.

### Targeting Material Separation in 2D Projections

Embodiments of the present disclosure allow straightforward identification of various tissue and synthetic materials and allow these materials to be distinguished from each other in projection image content. This identification of individual materials can then be used for subsequent 3D volume reconstruction. FIG. 6C shows a sequence that applies decomposition to 2D projection images to generate material-separated projection images. Reconstruction processing then uses the multi-channel volume data for generating 3D volume content.

Decomposition can also be provided according to user selection, as shown in the user interface example of FIG. 6D. One or more menu selections can provide the needed information for basis material and threshold definition used in material decomposition. Reconstruction can then be selectively applied to the desired portions of the multi-channel data from the material-separated projections.

### Exemplary Results—Root Canal Geometry Assessment

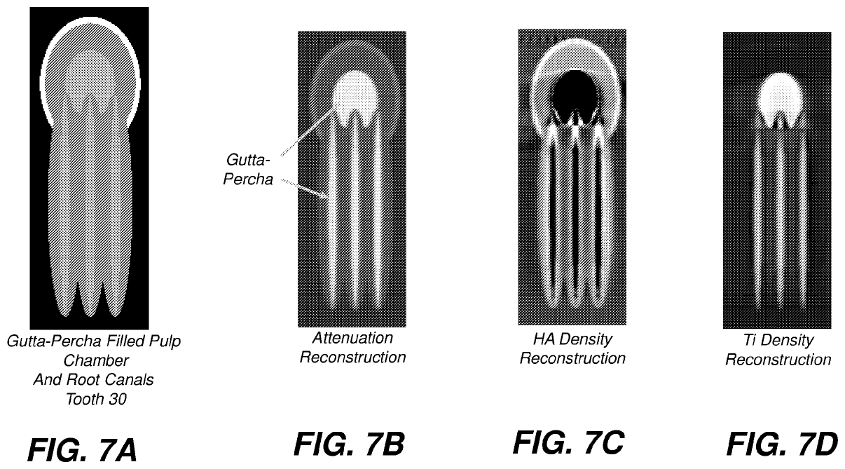

FIG. 7A shows a schematic for a dental phantom that is scanned using spectral x-rays in order to generate the 3D volume reconstructions represented in FIGS. 7B, 7C, and 7D. The phantom shows, for tooth 30, a pulp chamber packed with gutta-percha material and root canal structures having the same gutta-percha material. FIG. 7B shows a conventional 3D attenuation reconstruction from a volume imaging apparatus such as that shown in FIG. 1, obtained from a set of projection images acquired with x-rays at a single energy level. As is clearly shown, the imaging technique and x-ray energy used is optimized more closely for gutta-percha material, rather than for tooth or metallic material. Features and edges of the tooth structure are less distinct.

FIGS. 7C and 7D show examples of reconstructions generated when using spectral x-ray imaging techniques, with processing for two different basis materials. FIG. 7C shows a 3D volume reconstruction obtained with HA (hydroxyapatite), a material prevalent at different levels for tooth bone and enamel. FIG. 7D shows a reconstruction generated using titanium (Ti) as a basis material.

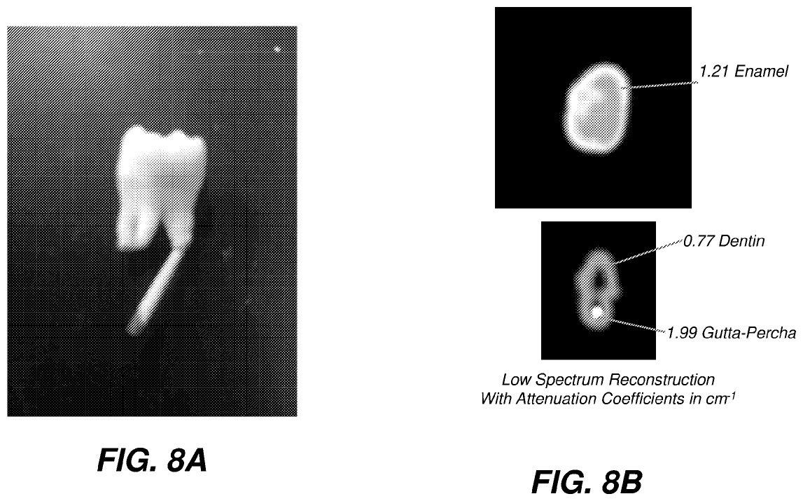

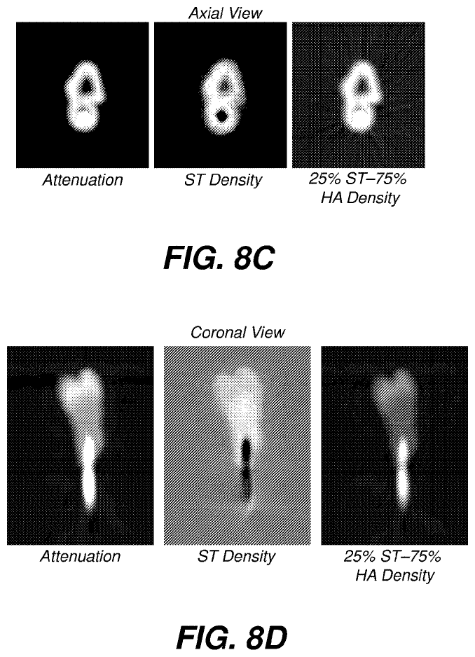

FIGS. 8A and 8B show examples for reconstructions from a tooth having gutta-percha material. The gutta-percha filling can be removed for assessment of root canal geometry as shown in the example images. FIG. 8A shows a photographic image of the tooth with inserted gutta-percha point for reference.

FIG. 8B shows axial views from a lower-energy spectrum reconstruction. The upper image in FIG. 8B shows a portion of the tooth showing tooth enamel with an associated attenuation coefficient, shown in units of cm⁻¹. Comparison of gutta-percha at 1.99 cm⁻¹ with dentin material at 0.77 cm⁻¹ is shown in the lower image in FIG. 8B.

FIG. 8C shows axial views from a reconstruction of the tooth of FIG. 8A, using different basis materials. The image at left is from overall attenuation, without consideration of photon energy thresholds. At center, the image reconstruction uses soft tissue (ST) as basis material. At right, the reconstruction employs the indicated percentages of ST and HA materials.

FIG. 8D shows coronal views from a reconstruction of the tooth of FIG. 8A, using the set of basis materials employed for FIG. 8C. The image at left is from overall attenuation, without consideration of photon energy thresholds. At center, the image reconstruction uses soft tissue (ST) as basis material. At right, the reconstruction employs the indicated percentages of ST and HA materials.

### Exemplary Results—Titanium Post Visualization

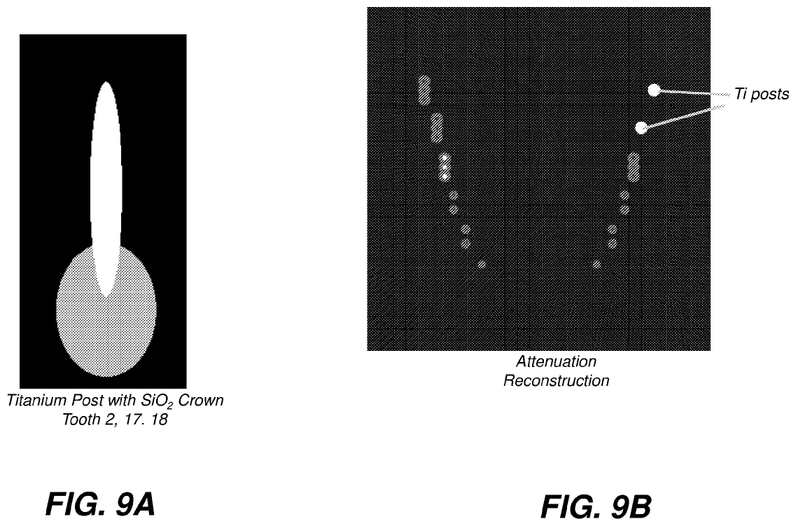

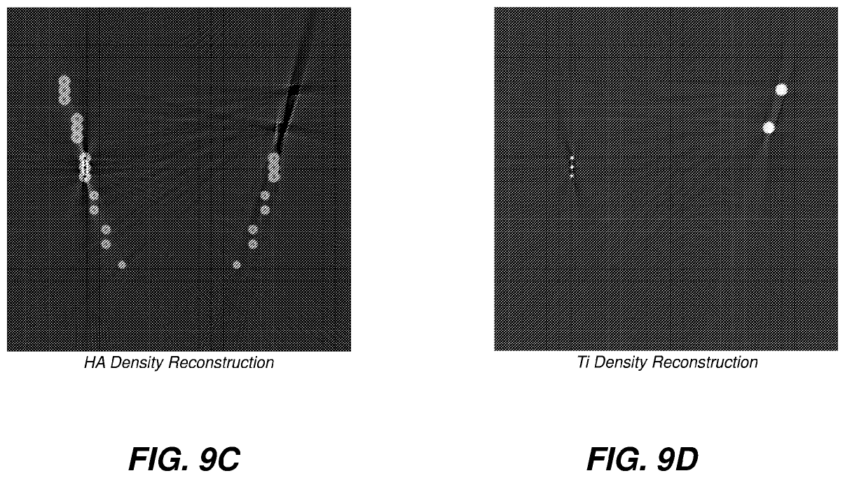

FIGS. 9A-9D show examples relating to segmentation and removal of titanium (Ti), such as would be useful for osseointegration assessment of an implant. Metal artifact reduction has been applied. FIG. 9A shows the phantom arrangement for a titanium post that lies partially within an SiO₂ crown. FIG. 9B is a conventional reconstruction based on attenuation over the full spectral range.

FIGS. 9C and 9D show exemplary reconstructions for representative Ti/SiO₂ structures in the phantom, using HA density and Ti density, respectively.

### Exemplary Results—Visualization for Tooth Structure Beneath Metal Crown

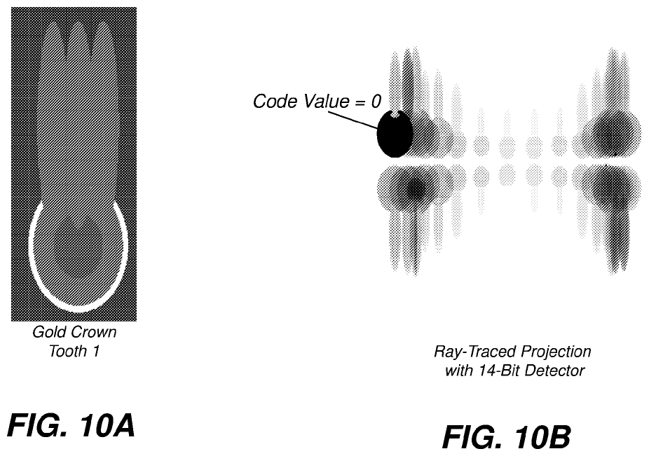

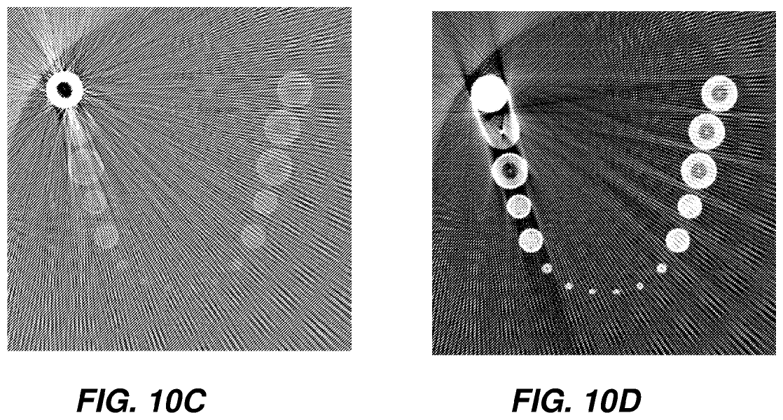

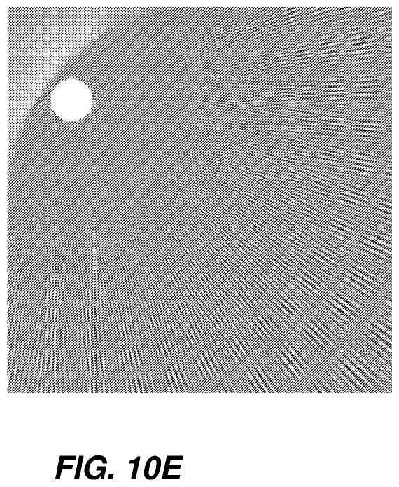

Due to its high atomic number (79), gold has very high photoelectric absorption, making it challenging to image crowns and other structures having gold content, as well as features adjacent to such structures. FIG. 10A shows the dental phantom arrangement used for imaging a gold crown positioned against tooth material.

FIG. 9B shows a ray-traced projection image for the gold crown of the phantom on tooth #1 at different angular orientations using a 14-bit detector.

FIG. 9C shows, in an axial view, a reconstruction of the phantom tooth with gold crown using basic attenuation reconstruction.

FIG. 9D shows a reconstruction of the phantom tooth using density of an HA basis material.

FIG. 9E shows a reconstruction of the phantom tooth using density of titanium as basis material.

FIGS. 10A-10D show images of reconstruction of a metal crown for an endodontic treatment. FIG. 10A shows a tooth with a metal crown. FIG. 10B shows an x-ray projection image for the tooth of FIG. 10A. FIG. 10C shows a reconstruction under HA density. FIG. 10D shows a reconstruction using Ti density. FIG. 10E shows a reconstruction of the phantom tooth using titanium as a basis material.

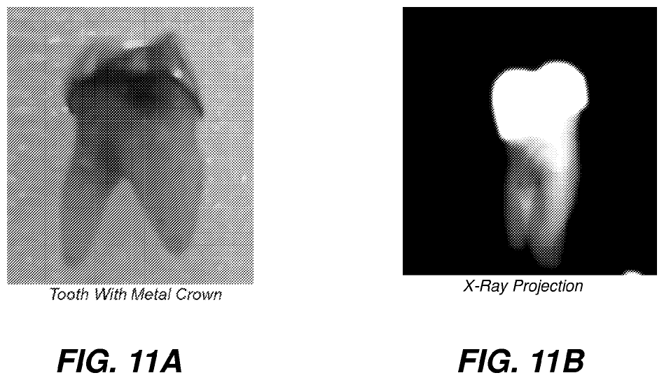

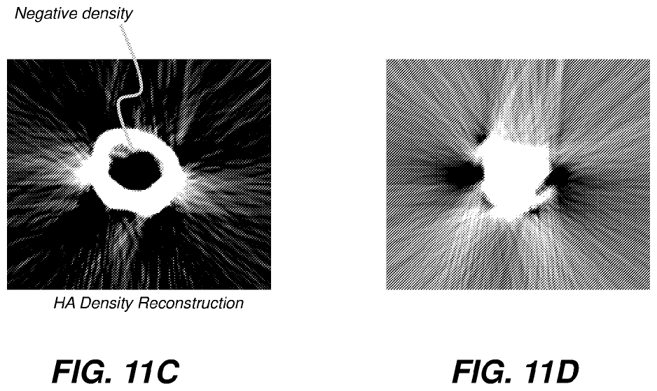

FIG. 11A shows a tooth with a metal crown.

FIG. 11B shows an x-ray projection image for the tooth of FIG. 11A.

FIG. 11C shows a reconstruction of a tooth using HA as a basis density. FIG. 11D shows a reconstruction of similar content using Ti density.

Exemplary Results—Visualization for Tooth Decay Assessment with Virtual Filling Removal

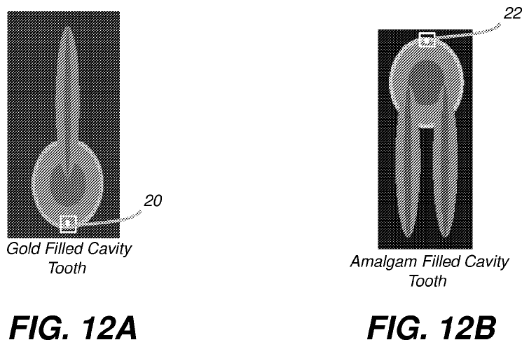

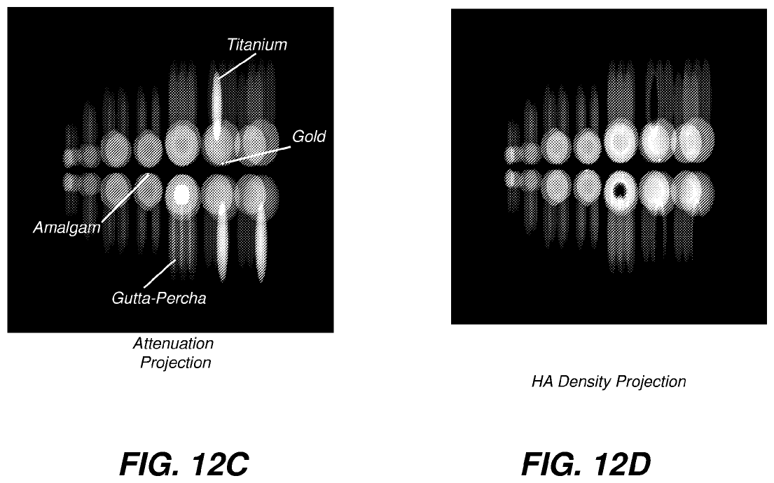

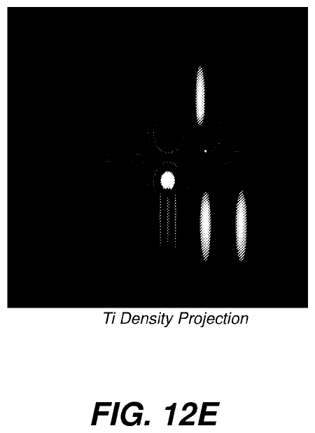

A useful feature of the method described herein relates to the ability to visualize filling material separately from its corresponding cavity structure and surfaces. FIG. 12A shows a tooth from an imaging phantom with a simulated gold-filled cavity 20 embedded within HA material. FIG. 12B shows a similar cavity 22 of amalgam.

FIG. 12C shows a reconstruction of phantom dentition, generated using overall attenuation, having both a gold filling and an amalgam filling, along with gutta-percha filling and titanium posts. As can be readily seen, features are presented but cannot be easily distinguished from surrounding tooth structures.

FIG. 12D shows a reconstruction formed using HA basis density. Features having densities of bone are pronounced and more readily distinguishable from metallic and other features.

FIG. 12E shows a reconstruction formed using Ti basis density, isolating gold and Ti features from lower density structures.

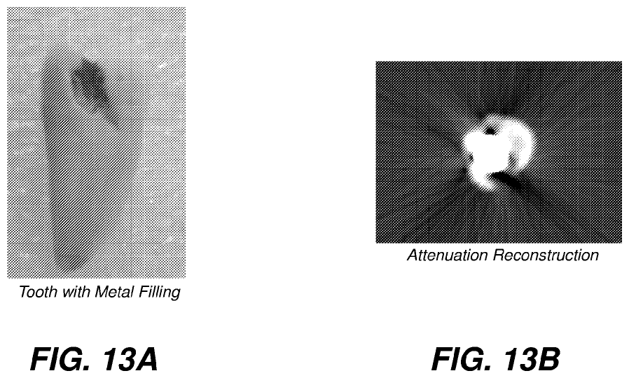

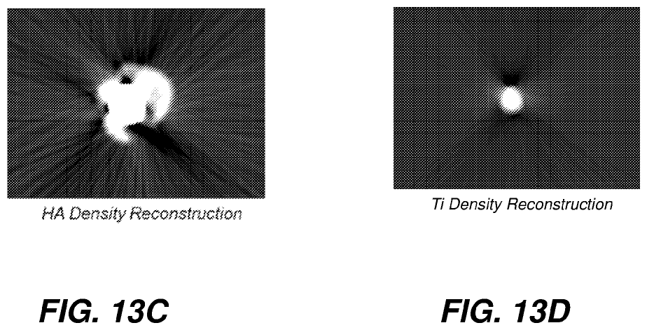

FIG. 13A shows a photograph of a tooth having a metal filling and imaged as related to different basis materials using methods of the present disclosure.

FIG. 13B shows a view of a reconstructed image of the tooth, generated using overall attenuation.

FIG. 13C shows a reconstruction of the tooth bony tissue using density of an HA basis material.

FIG. 13D shows a reconstruction of the metal filling using density of titanium as basis material.

### Exemplary Results—Soft Tissue Only Assessment

A benefit of the method of the present disclosure relates to enhanced capability for soft-tissue imaging, such as for root canal and pulp chamber assessment and for gum assessment.

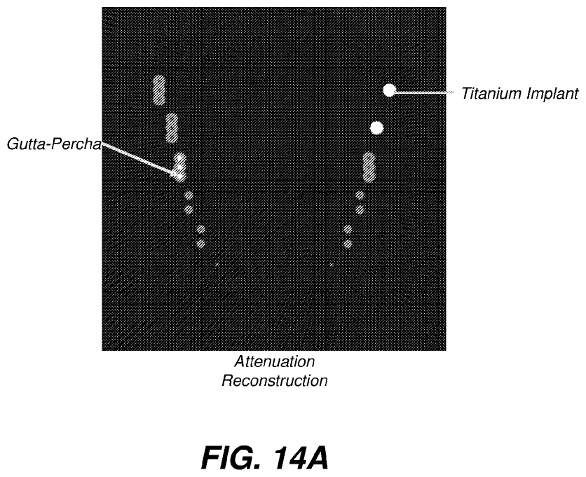

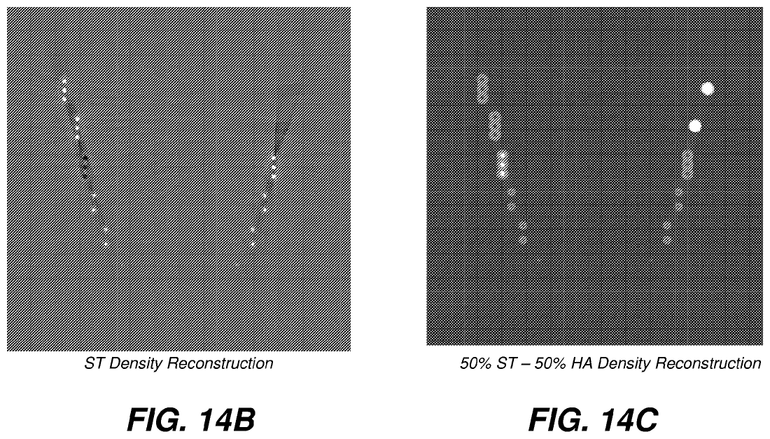

FIG. 14A shows a reconstruction using conventional attenuation imaging. FIG. 14B shows a reconstruction for soft tissue visualization using soft tissue (ST) basis material.

FIG. 14C shows a reconstruction for soft tissue visualization using a mix of ST and HA densities.

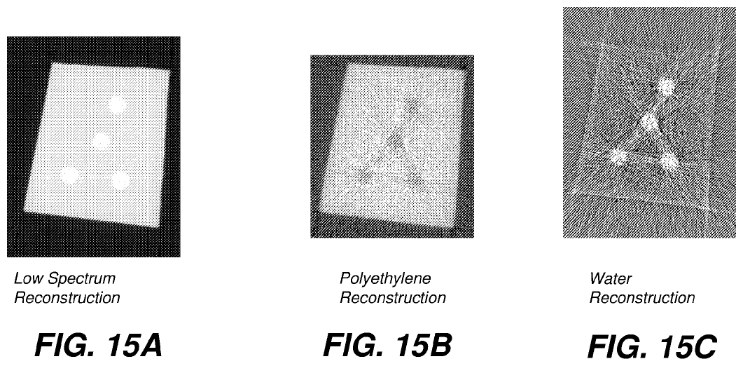

Gum assessment requires the capability for accurate and precise characterization of tissue density. Embodiments of the present disclosure are capable of more clearly distinguishing tissue densities as shown in the simulation examples of FIGS. 15A-15C. The subject is a polyethylene block having water-filled holes. FIG. 15A shows a reconstruction of the subject generated using lower-energy spectrum image content. FIG. 15B shows a reconstruction for the polyethylene component, with water removed from the image content.

FIG. 15C shows a reconstruction for the water component, segmented from the polyethylene block.

Spectral X-ray allows a virtual monochromatic reconstruction to be created at an energy at which material contrast is high. The difference in attenuation between the PE and water is 12% in the low energy spectrum reconstruction and 24% in the 30 kV virtual monochromatic range.

### Virtual Material Removal for Panoramic and Cephalometric Image Content

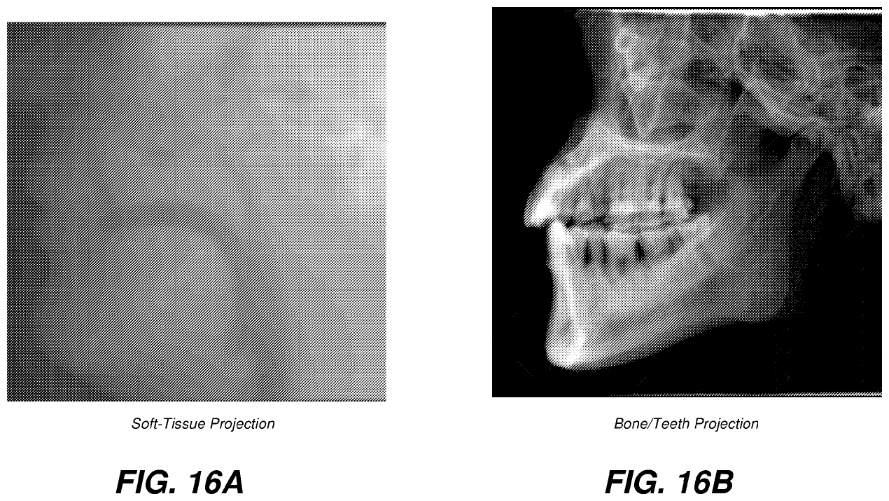

According to an embodiment of the present disclosure, virtual material removal methods can also be applied to panoramic and cephalometric image content. By way of example, FIG. 16A shows a projection image of a head phantom acquired using ST as a basis material. FIG. 16B shows a projection image of the head phantom acquired using 50% ST-50% HA composite. Combined results of ST and HA reconstruction are then displayed. Combination can include addition, weighting, averaging, and other methods for computing an image density value from a number of related values.

### User Interface Functions

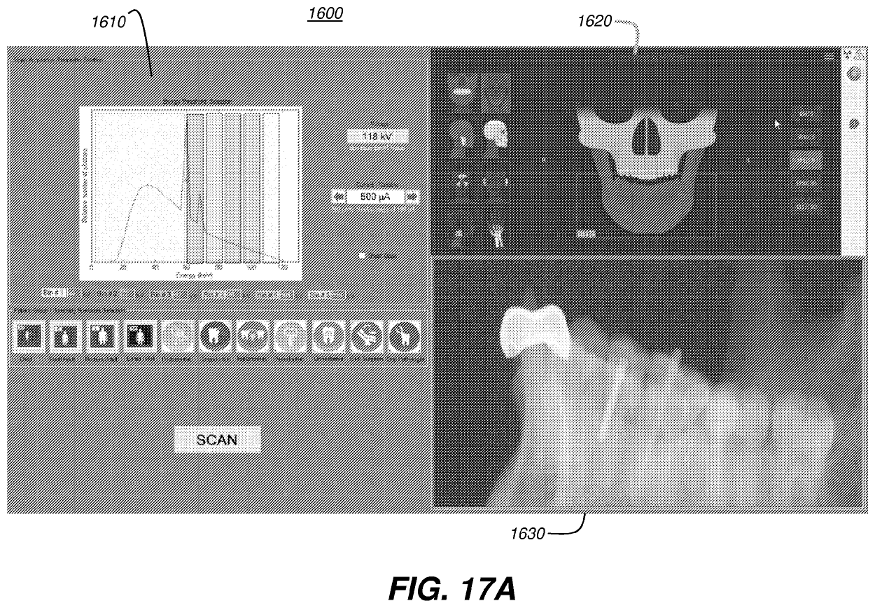

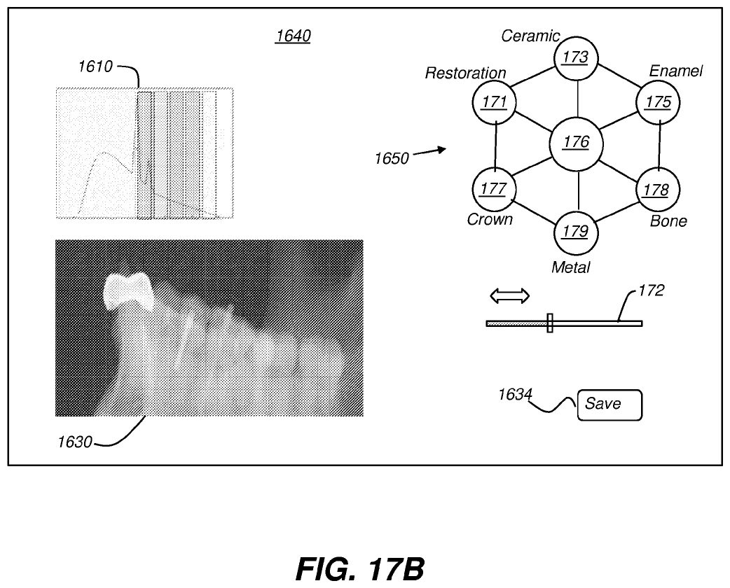

A graphical user interface (GUI) can be provided for configuring scan acquisition setup, decomposition variables, and display options, as shown in the examples of FIG. 17A and following.

FIG. 17A shows an exemplary acquisition parameters setup screen 1600 according to an embodiment of the present disclosure. A bin allocation section 1610 enables the user to set up an energy threshold (kV) for each of multiple energy bins for the photon counting system. Bin energy levels, as described previously, relate to base materials selected for exposure and subsequent decomposition functions. Screen 1600 can also display the tube voltage and allow adjustment of current values for the exposure apparatus.

A number of preset acquisition parameters can be available for selection, arranged according to patient size, age, or other characteristics or according to particular types of information needed by a particular specialist, as shown in the FIG. 16 screen. Thus, for example, separate sets of default parameters can be available for child or adult patients, or for periodontists, orthodontists, oral pathologists, oral surgeons, and other specialists. Presets can include bin threshold values, which may be adjustable by the operator, for example.

Acquisition parameters setup screen 1600 can also include an anatomy selection section 1620 that enables the operator to specify a region of the mouth or jaw that may be of particular interest. By mouse click or other selection mechanism, the operator selects particular anatomy, highlighted on the display. The region selected may be used by the system for adjustment of preset thresholds, for example. An optional display area 1630 can show the currently obtained image or a previous image of the area of interest specified by the operator.

### Channel Setup

As earlier shown, channels are used to provide a convenient tool for characterizing a large set of materials using a smaller set. The larger set of materials is found in the intraoral environment and includes not only various types of hard and soft tissue, but also numerous types of metals, ceramics, polymers, and composite materials. The smaller set of basis materials has well-known attributes relative to incident radiation across the x-ray spectrum.

Channels setup allows a material to be profiled according to spectral response over two or more basis materials. For an intraoral material, relative weighted values or percentages from two or more basis materials can not only be used for setting acquisition parameters, but also for decomposition processing and display functions.

A number of default channels are provided for typical combinations of basis materials. A user interface utility enables channel setup, prompting the user to specify a channel designation (for example, “7”, “gum tissue, adult”, “composite filling”) and a corresponding weighting, ratio, or percentage assigned to each of any number of basis materials (for example, HA: 8; ST: 2). The viewer can then store channel setups and edit channel setups as needed. As can be seen, channel parameters selection can be provided for various purposes, such as segmentation, tissue enhancement, gingival enhancement, and the like.

FIG. 17B shows an exemplary material parameters setup screen 1640 according to an embodiment of the present disclosure. Among its features, parameters setup screen 1640 gives the viewer the option to select different volume presentations and to modify not only the combinations of materials included, but also the relative intensity of each material within the volume, using the channel paradigm. Setup screen 1640 allows setup of a channel icon 1650 that can be used by a practitioner to adjust material parameters and to store a desired set of parameters and settings. Based on a patient chart, for example, a set of material selections 171, 173, 175, 177, 178, and 179 can be defined for a particular patient or condition. With an image shown in display area 1630, various material selections can be identified and selectively modified for rendering of similar anatomy features. Fine-tuning adjustment for enhancement of a particular material can be made by first identifying the material, such as by selection of one of the material selections 171, 173, 175, 177, 178, 179, such as using a mouse cursor or touchscreen. Once the particular material selection is made, use of an adjustment tool 172 such as a slide bar can provide the needed level of enhancement or suppression of image content for the selected material. This adjustment can be carried out for each of the various materials, using an image displayed in display area 1630 as a guide to the resultant image rendering. A Save button 1634 can be provided to allow a practitioner the option to name and save a channel setup with customized settings 176 suitable for a particular patient or particularly suited to a practice or specialty. Newly saved channel setups can be displayed with channel setups previously set up in manufacture, for example.

### Image Acquisition and Decomposition Using Channel Function

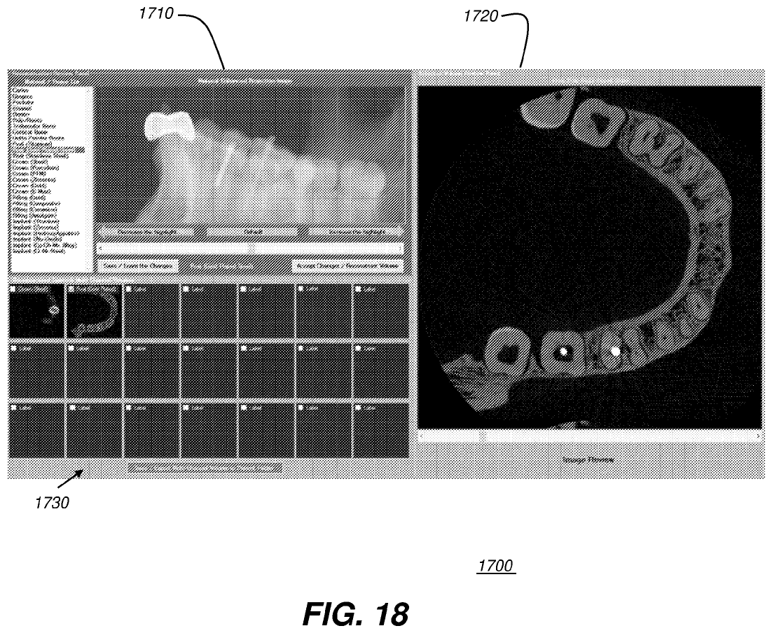

FIGS. 18, 19, 20, and 21 show various ways the channel structure can be used for materials decomposition and to modify image reconstruction and display for visualizing different materials represented in the acquired image content.

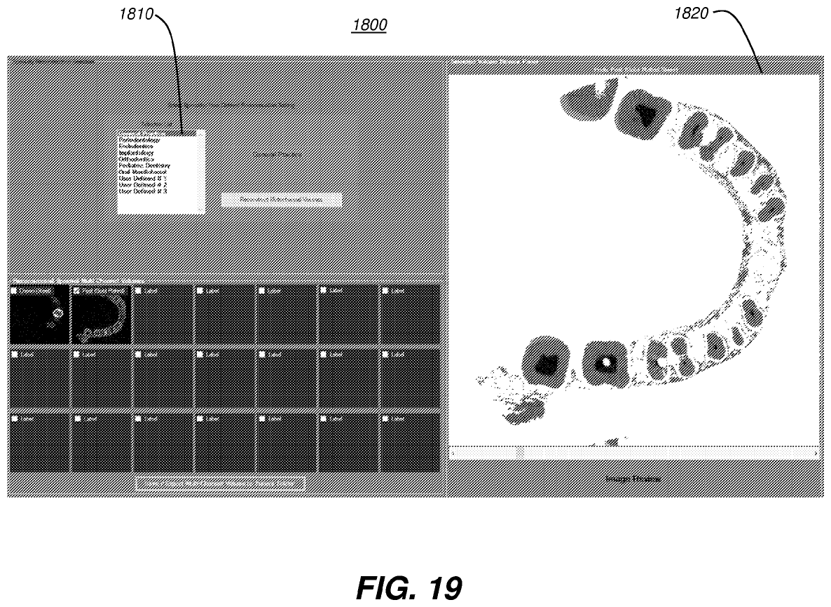

FIGS. 18 and 19 show a decomposition selection screen 1700, 1800 for choosing aspects of projection decomposition at a detailed level. A 2-D reconstruction review panel 1710 can provide an image of the reconstructed 2-D projection with a menu listing of basis materials that can be applied to the reconstruction algorithms. Further controls can allow adjustment of highlight effects. A 3-D volume reconstruction review panel 1720 can then show a representative image volume reconstructed according to the selected projection variables. An image storage area 1730 stores temporary results from operator manipulation of patient image content.

The decomposition selection screen 1800 of FIG. 19 also provides a work area for projection decomposition in an alternate format. A selection menu 1810 allows operator selection of decomposition parameters according to specialty, such as orthodontics, endodontics, periodontics, and the like. In addition, menu selection can also be provided for one or more user-defined parameters sets, including parameters defined in previous exams, for example. A 3-D volume reconstruction panel 1820 can then show a representative image volume reconstructed according to the selected projection variables. Advantageously, multiple volumes can be stored with different material decomposition factors, allowing a single scan to provide a substantial amount of data on the material composition of oral anatomy features for the patient.

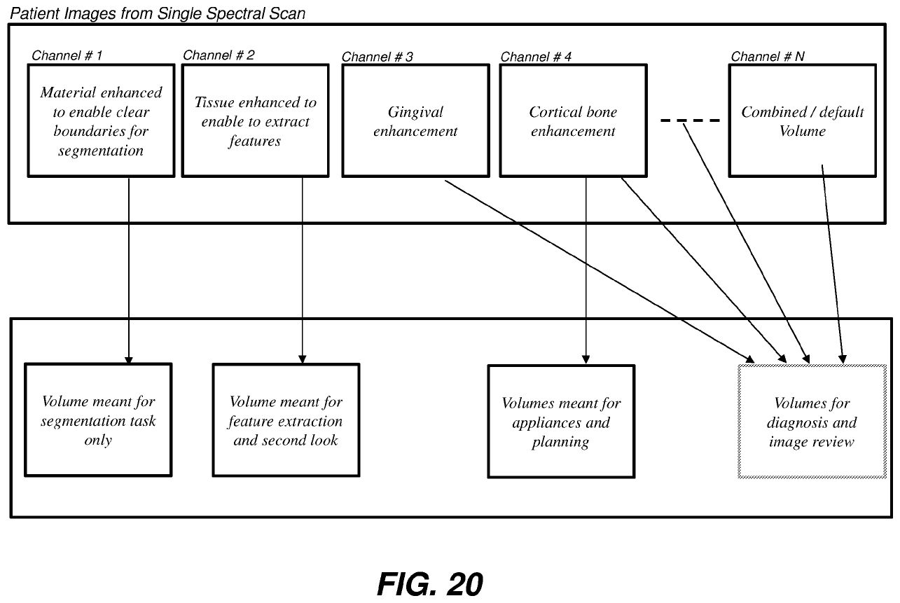

FIG. 20 shows views of multiple channels that can be applied to a single spectral scan. Channels can be used individually or in combination in order to help visualize the acquired image content, as 2D images or as a 3D volume reconstruction. As shown in FIG. 20, a channel #1 is used to show segmentation boundaries more clearly. A channel #2 shows tissue enhanced to enable clear identification and extraction of key features. A channel #3 provides enhancement of gingival tissues. A channel #4 provides enhancement of cortical bone features. Another channel can provide a combined volume as a default.

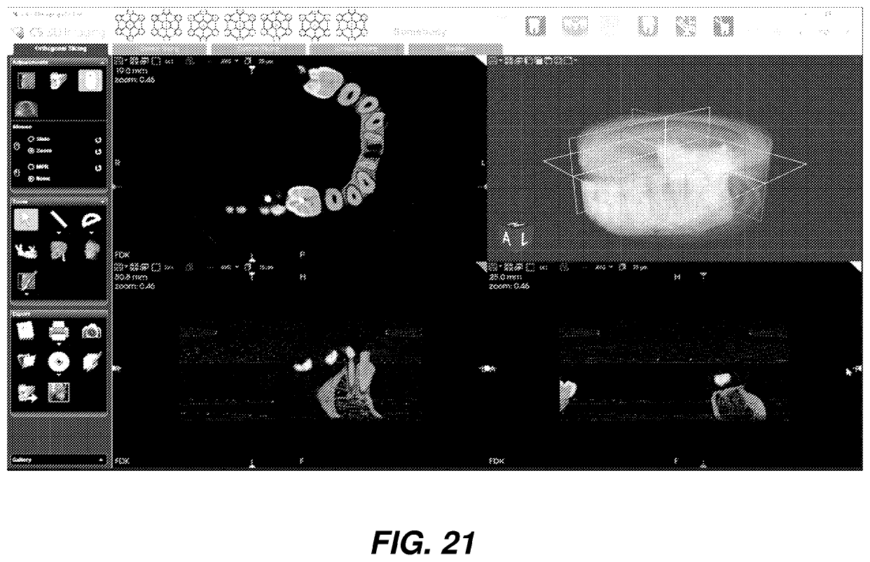

FIG. 21 shows a composite user interface display allowing channel selection and different views of the acquired image content. FIG. 21 also shows alternative views of a spectral volume that can be generated according to an embodiment of the present disclosure. This can include 2-D projection views or 3-D reconstructed volumes from the same acquisition scan, using any combination of material decomposition factors. A number of display tools can be provided for view angle and rotation, zoom, measurement. A numbered channel icon allows selection for viewing a particular volume.

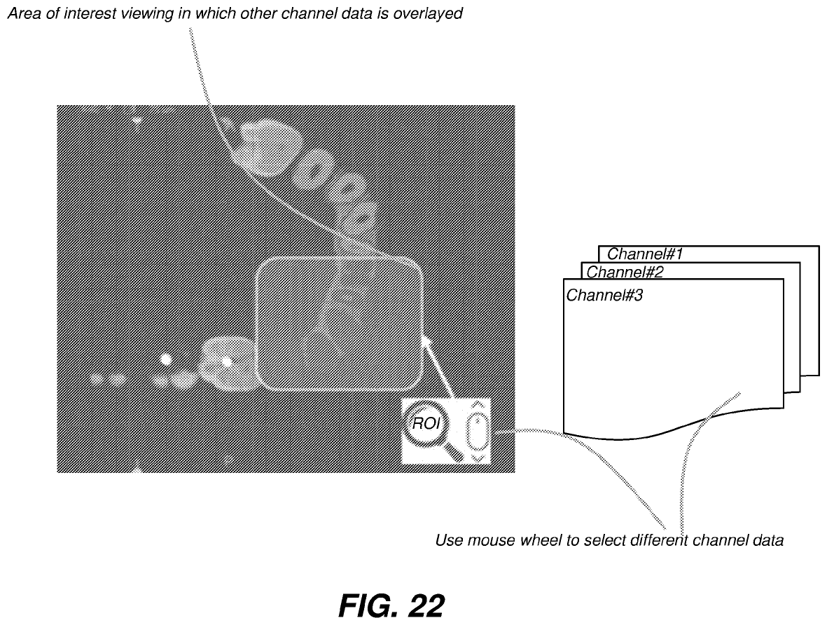

FIG. 22 shows an operator interface using a mouse or other pointer to scroll through different views of the data by channel number.

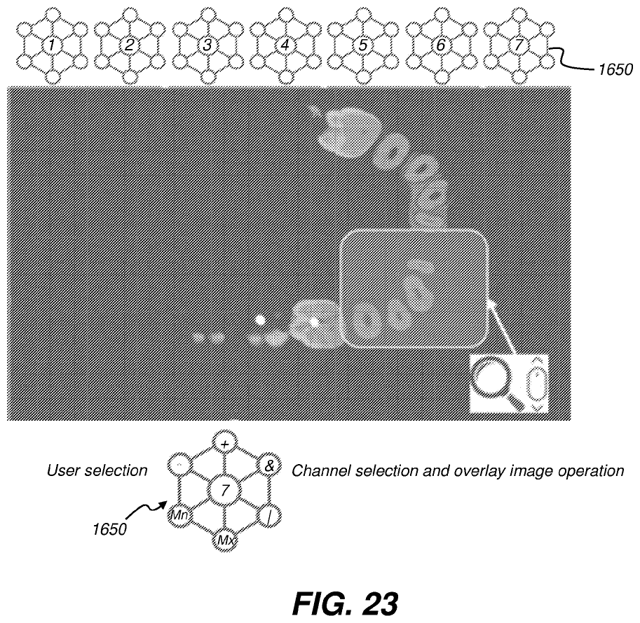

FIG. 23 shows a portion of an operator interface display that can provide an alternate method of user channel selection and combination using icons 1650. Seven channels are listed, by way of example, in channel icons 1650. The operator can select one or more channels for display, such as using mouse cursor or touchscreen selection, or using the mouse wheel. For each selected channel, a number of logical operations are provided for combination, such as addition, subtraction, AND, OR, Max., and Min, functions, for example. This user interface arrangement, more complex than the arrangement for channel adjustment shown in FIG. 17B, allows multiple channel combinations of channels to support the viewer, such as with channels added, subtracted, or combined using other logical operations as shown.

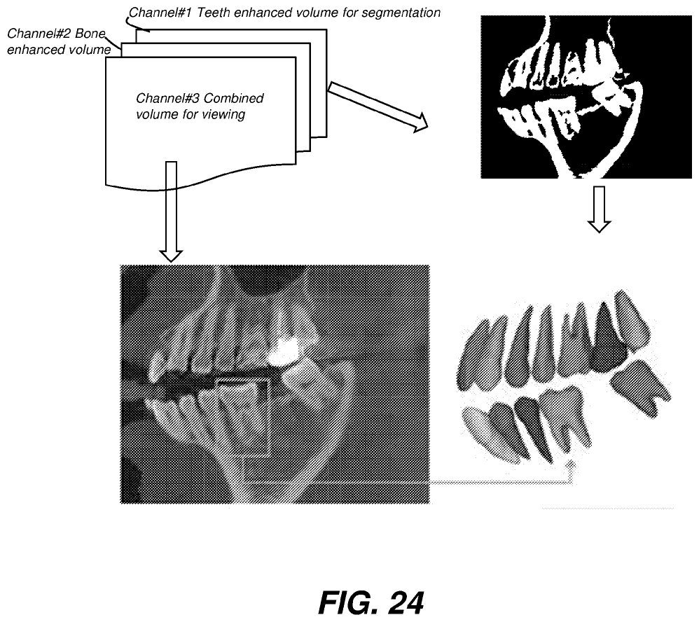

FIG. 24 shows how channel parameters selection can be used to provide improved segmentation. For example, segmentation can effectively distinguish tooth components from gum tissue and from surrounding alveolar bone structures. A channel #1 provides a view of teeth enhanced for segmentation. A channel #2 shows enhanced bone volume. A channel #3 provides the full volume view.

### Virtual Removal

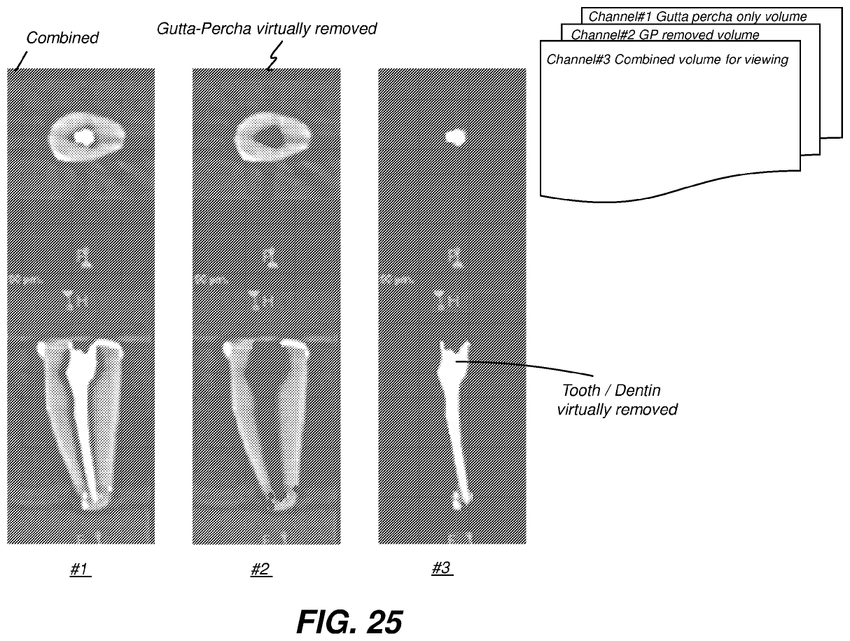

Virtual removal capability allows enhanced visualization of selected portions of the image content according to material composition. With reference to FIG. 25, selection of specific channels enables visualization, or removal, of particular materials in the presented 2D or 3D views. Removal can be equivalent to reducing opacity to minimal levels or to effectively zero. This can include virtual material removal of crown, gutta percha and post material, for example, from the oral anatomy, allowing the viewing practitioner to assess the living tissue interface and surfaces surrounding the restorations. Features formed from man-made or fabricated materials not native to the intraoral or oral environment can be distinguished from tissue in the imaged oral anatomy and from supporting anatomical structures.

At left in FIG. 25 is a combined image, available with channel #1 setup, having material components with various attenuation characteristics. A second image, corresponding to channel #2 setup, is conditioned to provide a view of the image content with the gutta-percha component removed from the image. A third channel setup, channel #3 in FIG. 25, shows conditioning of image content by tooth and dentin removal, thus showing gutta-percha features more clearly.

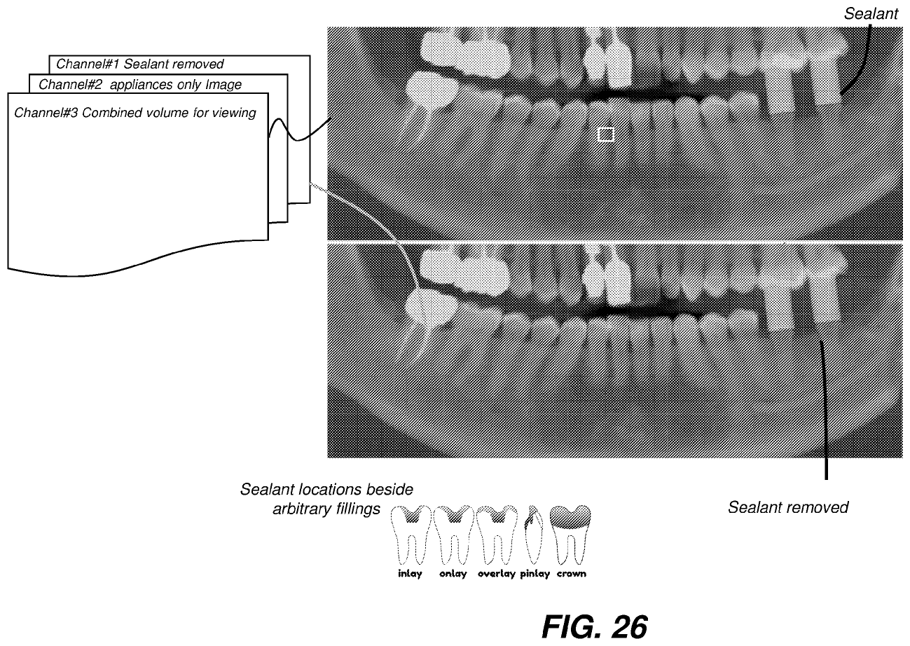

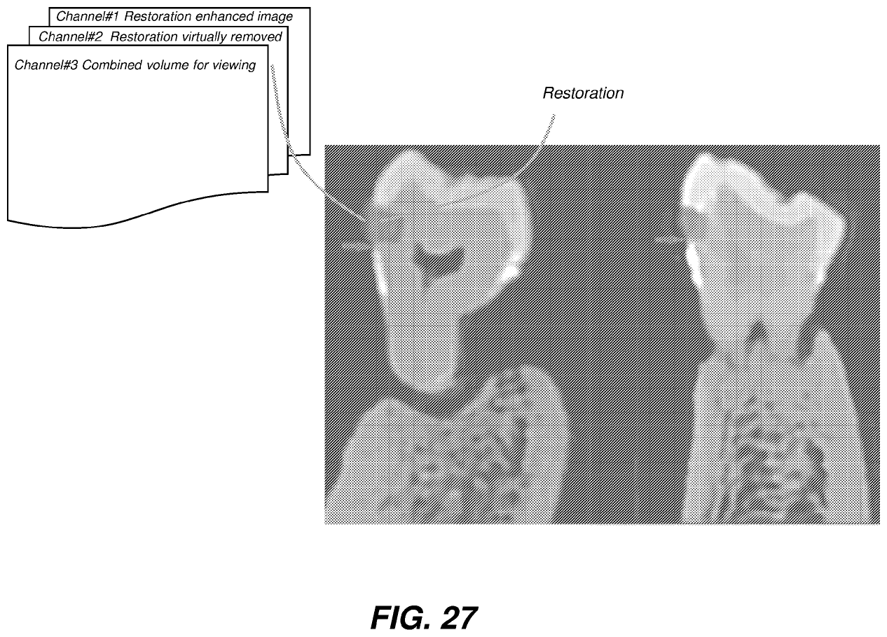

FIG. 26 shows virtual removal of sealant material that had been deposited upon the dentition. Removal of sealant content can enable the assessment of healing processes for the affected teeth. In the example figure shown, a channel #1 provides a view with sealant removed. Channel #2 provides an image with dental appliances only and other features removed. Channel #3 shows a combined image that allows visibility of teeth, dental appliances, and sealant features. FIG. 26 shows exemplary sealant locations relative to arbitrary fillings.

FIG. 27 shows virtual removal of restorative material. This feature allows the viewing practitioner to more clearly visualize tooth fractures or decay. Channel #1 shows a restoration-enhanced image. Channel #2 shows the image with restoration content virtually removed. Channel #3 shows the combined volume, including restoration features. Typical restorative materials can include gold, porcelain, composite resins, amalgam, ceramics, and zirconia, for example.

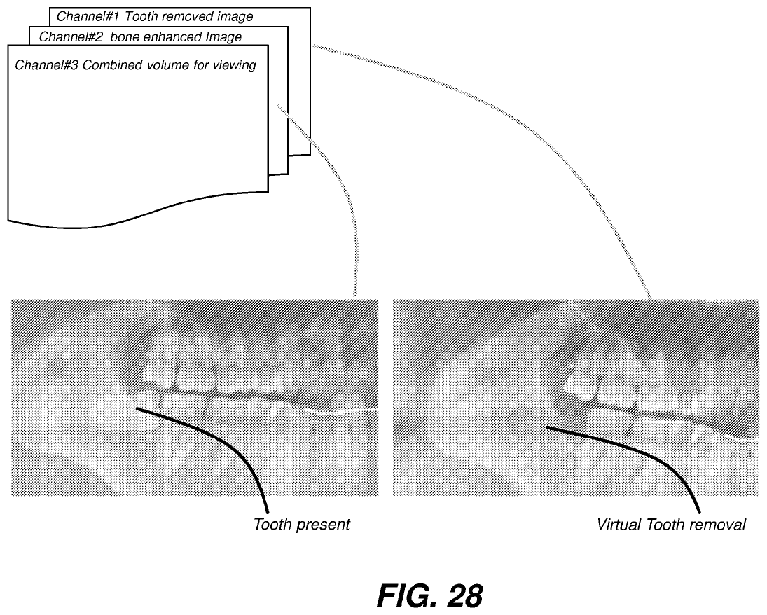

FIG. 28 shows an example of virtual tooth extraction. This feature enables improved visibility of nearby supporting structures and can be useful prior to surgical procedure. Selection of channel #1 enables the decomposition software to isolate a tooth from surrounding features. Channel #2 can provide a bone-enhanced image. Channel #3 can provide a combined volume that shows the tooth and nearby features.

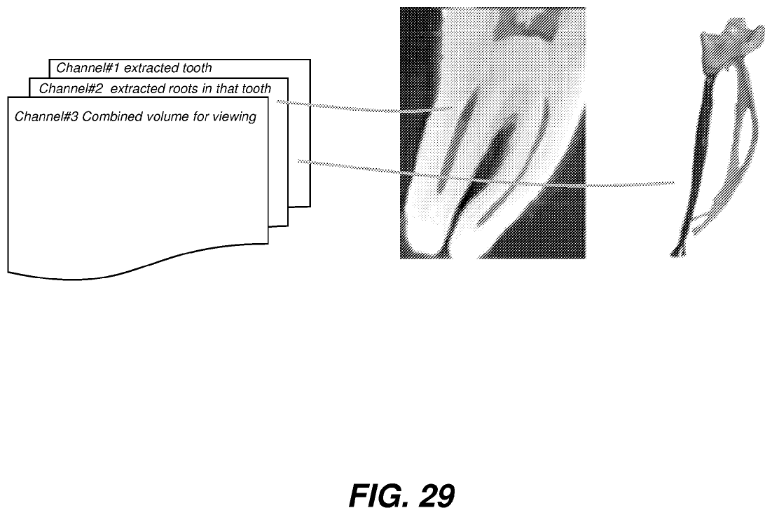

FIG. 29 shows an example with virtual tooth extraction, as an aid to root canal treatment. Channel #1 selection shows the tooth content only. Channel #2 shows only the extracted root material. Channel #3 shows a combined volume.

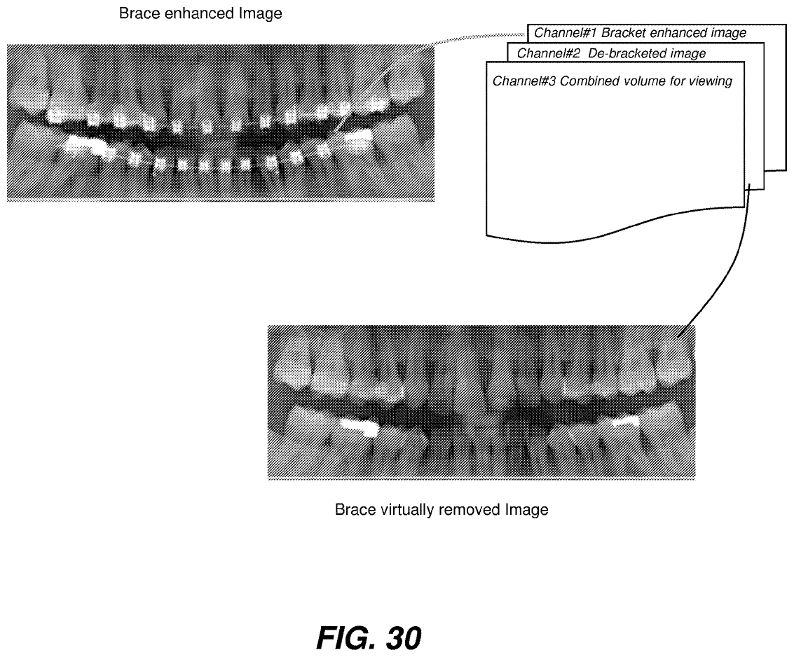

FIG. 30 shows virtual brace removal in orthodontic treatment. Channel #1 selection provides a bracket-enhanced image. Channel #2 shows an image with bracket contents suppressed or removed. Channel #3 shows a combined volume for viewing. Brackets that can be identified can include traditional metal braces, including brackets, ceramic braces, including color brackets, lingual braces, brackets on the back of teeth or facing the tongue, and clear aligners, for example.

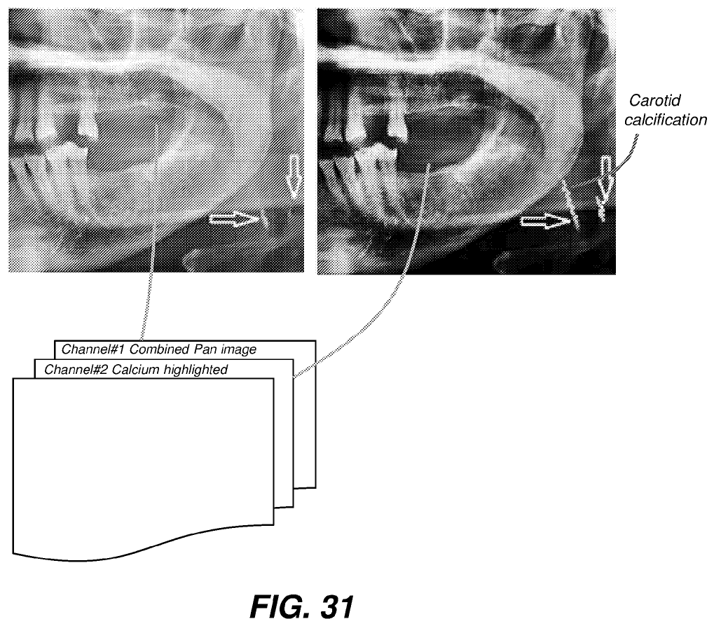

FIG. 31 shows a display of separated or decomposed calcifications and soft tissue, with channel setup useful for highlighting carotid calcification and tumors. Channel #1 selection provides a combined panoramic image. Channel #2 selection shows an image with calcium contents enhanced.

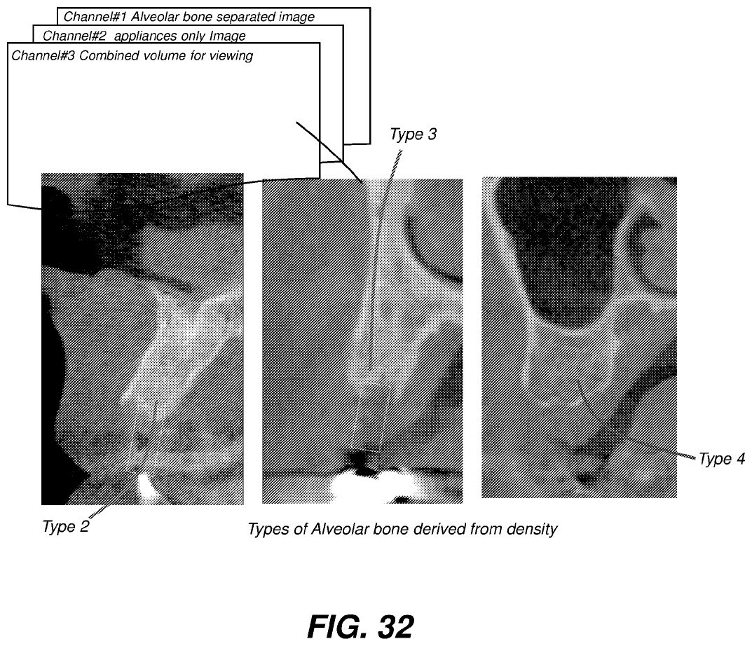

FIG. 32 shows displayed material separation, with highlighting of alveolar bones to help in assessing and qualifying alveolar bone structures for implant material. Bone types can be identified in the acquired image content, as shown. Channel #1 selection provides an alveolar bone-separated image. Channel #2 selection yields an appliance-only image. Channel #3 provides a combined volume for viewing.

Bone quality can be categorized by group:
Type I—Homogeneous cortical bone.
Type II—Thick cortical bone with marrow cavity.
Type III—Thin cortical bone with dense trabecular bone of good strength.
Type IV—Extremely slim cortical bone with low-density trabecular bone of poor strength.

### Assessment and Planning

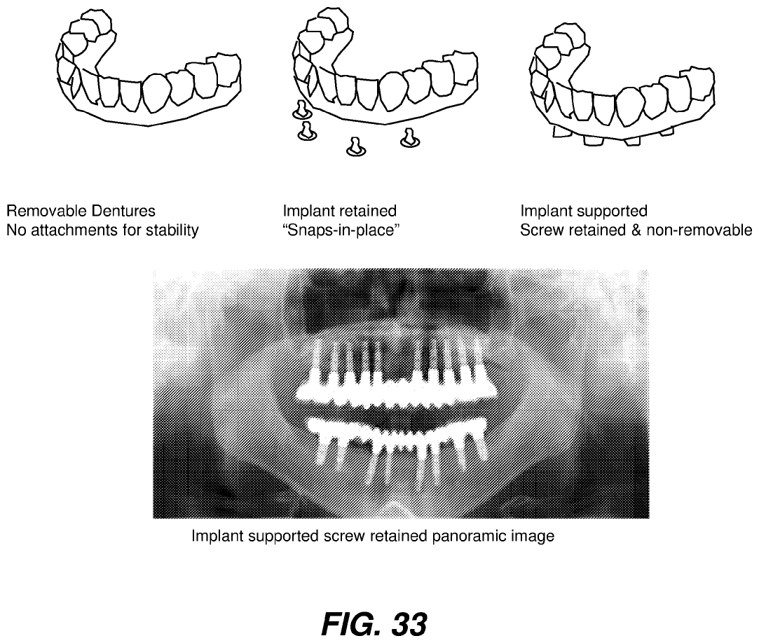

FIG. 33 is a diagram showing virtual removal of dentures and surgical guides in restorative dentistry.

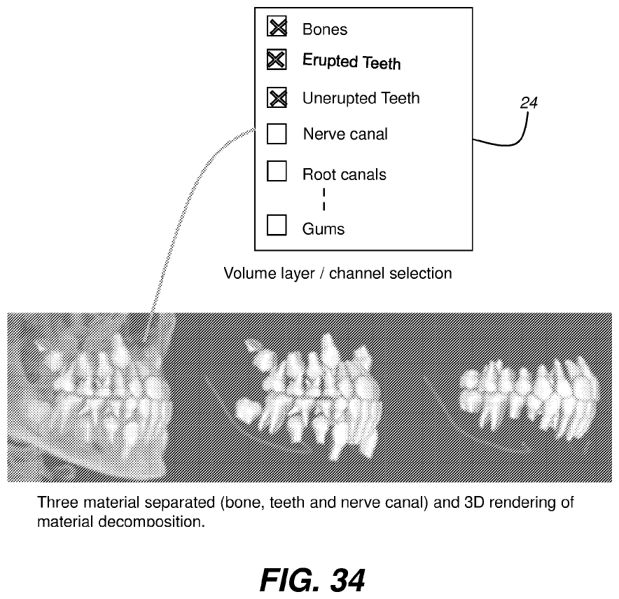

FIG. 34 shows different 3D renderings by material for various types of anatomy. A user interface checklist 24 provides a utility for selection of materials for display.

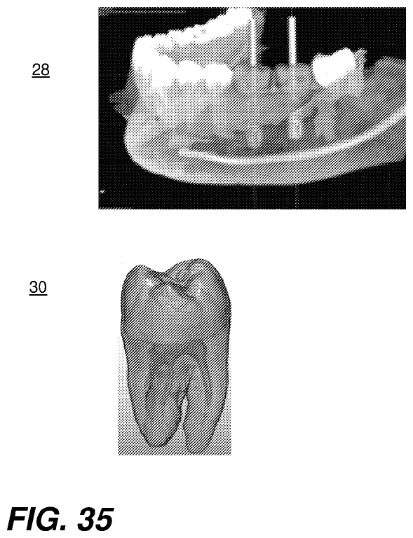

FIG. 35 shows an exemplary 3D rendering 28 of separate components, including teeth, alveolar bone, implant structures, nerve canal, and crowns. Another 3D rendering 30 shows dentin, crown, and root separated, within a reconstructed volume.

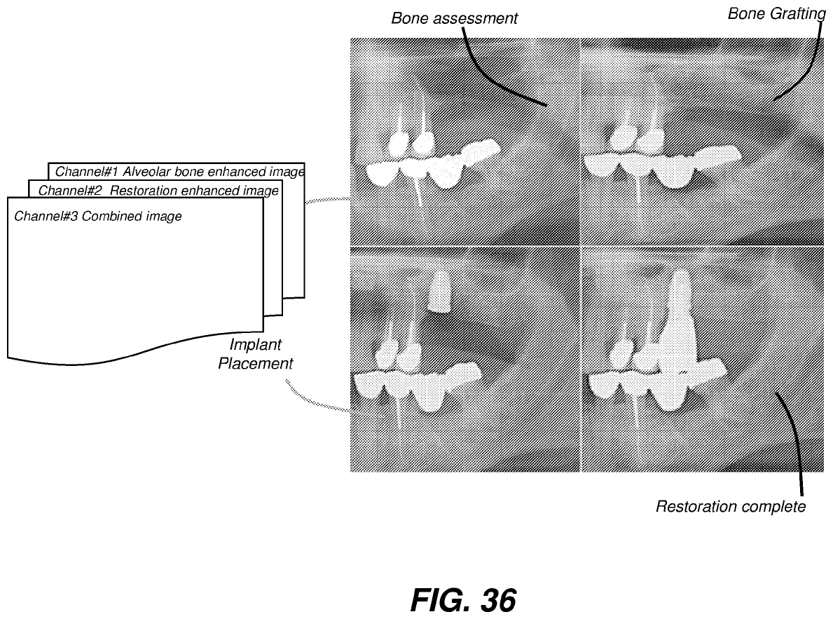

FIG. 36 is a diagram showing use of spectrally enhanced images in bone grafting for restoration and implant procedures. This capability can be used for evaluation of socket grafts, block bone graft, or sinus lift procedure, for example. A channel #1 provides a bone-enhanced image. A channel #2 provides a restoration-enhanced image. A channel #3 provides a combined image.

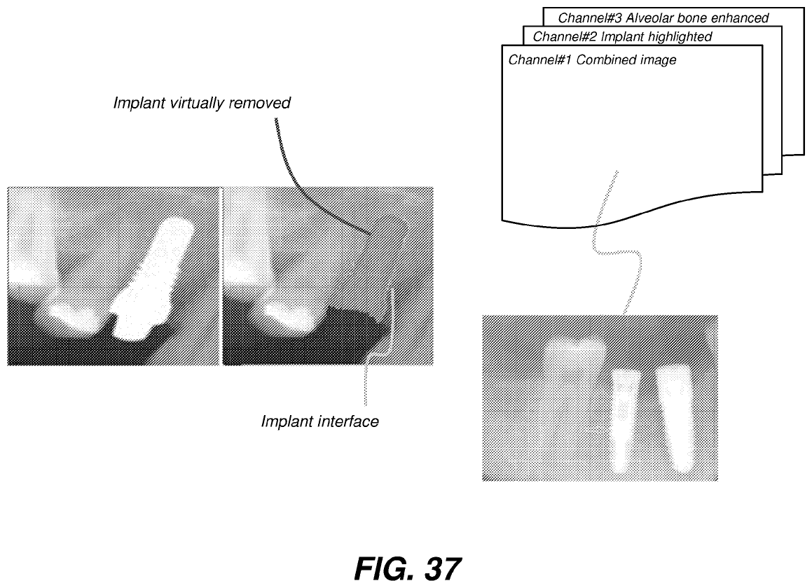

FIG. 37 shows imaging for osseointegration assessment. Virtual removal of the implant from surrounding alveolar bone tissues allows a more accurate assessment of the osteointegration process.

The presently disclosed exemplary embodiments are therefore considered in all respects to be illustrative and not restrictive. The scope of the disclosure is indicated by the appended claims, and all changes that come within the meaning and range of equivalents thereof are intended to be embraced therein.

Consistent with at least one exemplary embodiment, exemplary methods/apparatus can use a computer program with stored instructions that perform on image data that is accessed from an electronic memory. As can be appreciated by those skilled in the image processing arts, a computer program of an exemplary embodiment herein can be utilized by a suitable, general-purpose computer system, such as a personal computer or workstation. However, many other types of computer systems can be used to execute the computer program of described exemplary embodiments, including an arrangement of one or networked processors, for example.

A computer program for performing methods of certain exemplary embodiments described herein may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. Computer programs for performing exemplary methods of described embodiments may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the application, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that can be directly associated with a display device and is periodically refreshed as needed in order to provide displayed data. This temporary storage buffer can also be considered to be a memory, as the term is used in the application. Memory is also used as the data workspace for executing and storing intermediate and final results of calculations and other processing. Computer-accessible memory can be volatile, non-volatile, or a hybrid combination of volatile and non-volatile types.

It will be understood that computer program products for exemplary embodiments herein may make use of various image manipulation algorithms and/or processes that are well known. It will be further understood that exemplary computer program product embodiments herein may embody algorithms and/or processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the application, are not specifically shown or described herein and may be selected from such algorithms, systems, hardware, components and elements known in the art.

Exemplary embodiments according to the application can include various features described herein (individually or in combination).

While the invention has been illustrated with respect to one or more implementations, alterations and/or modifications can be made to the illustrated examples without departing from the spirit and scope of the appended claims. In addition, while a particular feature of the invention can have been disclosed with respect to only one of several implementations/exemplary embodiments, such feature can be combined with one or more other features of the other implementations/exemplary embodiments as can be desired and advantageous for any given or particular function. The term “a” or “at least one of” is used to mean one or more of the listed items can be selected. The term “about” indicates that the value listed can be somewhat altered, as long as the alteration does not result in nonconformance of the process or structure to the illustrated exemplary embodiment. Finally, “exemplary” indicates the description is used as an example, rather than implying that it is an ideal. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for forming radiographic images of oral anatomy of a subject comprising:
a) exposing the subject to radiation along an x-ray radiation path;
b) obtaining image content by counting the number of photons received, on a detector array in the radiation path, from the radiation through the subject at each of a plurality of positions,
wherein the detector maintains a first photon count for photons having a first energy above a first threshold energy value and further maintains at least a second photon count for photons having a second energy above a higher threshold energy value;
c) identifying one or more materials in the mouth of the subject by performing material decomposition processing, according to two or more basis materials that have been previously characterized according to the at least first and second photon counts, and distinguishing between natural oral anatomy features and fabricated materials in the mouth of the subject according to the material decomposition processing;
and
d) displaying conditioned image content that enhances the distinction between the identified natural oral anatomy features and fabricated materials in the mouth according to the material decomposition.

2. The method of claim 1 further comprising accepting an operator-entered instruction specifying one or more basis materials for material decomposition.

3. The method of claim 1 wherein the conditioned image content decreases the opacity of one or more of the natural oral anatomy features and fabricated material in the mouth.

4. The method of claim 1 wherein the image content is a 3D reconstruction formed from a set of two-dimensional radiographic images acquired over a set of projection angles about the subject.

5. The method of claim 1 wherein the image content is a 2D projection image.

6. The method of claim 1 wherein the basis materials are taken from the group consisting of a fluid, hydroxyapatite, and a metal.

7. The method of claim 1 further comprising accepting the at least the first threshold energy value entered by a user.

8. The method of claim 1 wherein the detector further accumulates photon energy lying between the higher threshold energy value and a third threshold energy value.

9. The method of claim 1 further comprising responding to a user instruction to modify one or both of the at least the first and higher threshold energy values.

10. The method of claim 1 wherein the first threshold energy value is obtained using materials information from a patient's dental chart.

11. The method of claim 1 wherein the first threshold energy value is obtained from an initial image of the subject.

12. The method of claim 1 further comprising setting exposure values for x-ray radiation according to an initial image.

13. The method of claim 1 wherein the first threshold energy value is a predetermined value.

14. The method of claim 1 wherein displaying conditioned image content comprises enhancing visibility of features, within the image, of a first material and suppressing visibility of features of a second material.

15. The method of claim 14 wherein enhancing and suppressing are controlled on a user interface display.

16. The method of claim 14 wherein the first material is a natural feature of the subject anatomy and the second material is fabricated.

17. The method of claim 16 wherein the second material has a k-edge for attenuation within the spectral range of the radiation.

18. The method of claim 1 wherein the fabricated material forms a root canal filling.

19. The method of claim 1 wherein the fabricated material forms an implant.

20. The method of claim 1 wherein the fabricated material is a sealant.

21. The method of claim 1 wherein the fabricated material is a dental filling.

22. The method of claim 1 wherein the fabricated material is a restoration material.

23. The method of claim 1 wherein the fabricated material forms a dental crown.

24. The method of claim 1 wherein the fabricated material forms a denture.

25. The method of claim 1 wherein displaying conditioned image content according to the material decomposition further comprises responding to a user selection of a channel, wherein the channel identifies a combination of one or more basis materials, and corresponding densities, used for material decomposition processing.

26. A method for forming a radiographic image of oral anatomy of a subject comprising:
a) exposing the subject to x-ray radiation;
b) acquiring image content by obtaining photon energy from the radiation through the subject at a detector array that accumulates, at each of a plurality of array positions, a first photon count for photons having a first energy above a first threshold energy value and at least a second photon count for photons having a second energy above a higher threshold energy value;
c) forming conditioned image content by performing material decomposition processing on the acquired image content according to the at least first and second photon counts, wherein the material decomposition distinguishes between natural oral anatomy features and fabricated materials in the mouth of the subject, relative to two or more basis materials that have been characterized according to the at least first and second photon counts;
d) storing the conditioned image content;
e) identifying at least a first and a second material within the imaged oral anatomy according to the conditioned image content;
f) repeating steps a) through c) at a range of angles about the subject; and
g) reconstructing and displaying a 3D image of the subject from the stored image content.

27. The method of claim 26 further comprising highlighting at least the first material within the reconstructed 3D image.

28. The method of claim 26 further comprising selecting the one or more basis materials according to patient chart data.

29. The method of claim 26 further comprising selecting the one or more basis materials according to an initial scout image.

30. The method of claim 26 wherein performing the material decomposition process comprises responding to an operator selection of a channel that defines a set of basis materials.

31. The method of claim 30 further comprising storing the reconstructed 3D image according to the selected channel.

32. The method of claim 26 wherein performing the material decomposition processing comprises responding to an operator specification of a dental specialty.

33. The method of claim 26 further comprising presenting alternate views of the conditioned image content according to attenuation properties of imaged materials.

34. The method of claim 30 wherein a first channel selection shows an image with sealant features visible and a second channel selection shows an image with sealant features transparent.
